SEQUENCE LISTING (24 sequences)

SEQ ID NO: 1 - protein, 2016 aa, Homo sapiens [sequence omitted]

SEQ ID NO: 2 - DNA, 8504 bp, Homo sapiens
agacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatga

SEQ ID NO: 3 - DNA, 8504 bp, Homo sapiens
agacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcgtatgtatcagaaaatataaaactaggcaatttgtcggctcttcgaactttcagagtcctgagagctctaaaaactatttcagttatcccagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatga

SEQ ID NO: 4 - DNA, 20 bp, Artificial
  Primer
ctgcacgcgttcactttcct

SEQ ID NO: 5 - DNA, 24 bp, Artificial
  Primer
gacaaattgcctagttttatattt

SEQ ID NO: 6 - DNA, 20 bp, Artificial
  Primer
ctgcacgcgttcactttcct

SEQ ID NO: 7 - DNA, 21 bp, Artificial
  Primer
cagccagcttcttcacagact

SEQ ID NO: 8 - DNA, 20 bp, Artificial
  Primer
tatacacccatctccagcga

SEQ ID NO: 9 - DNA, 20 bp, Artificial
  Primer
catctcctcattcacgaagc

SEQ ID NO: 10 - DNA, 20 bp, Artificial
  Primer
tgggaacaagagggcatctg

SEQ ID NO: 11 - DNA, 22 bp, Artificial
  Primer
ccaccactgcatcaaattcatg

SEQ ID NO: 12 - RNA, 19 nt, Artificial
  siRNA
agguaguguaaucgccuug

SEQ ID NO: 13 - RNA, 19 nt, Artificial
  siRNA
cuaggcaauuugucggcuc

SEQ ID NO: 14 - RNA, 19 nt, Artificial
  siRNA
uaucauggcguauguauca

SEQ ID NO: 15 - RNA, 19 nt, Artificial
  siRNA
gaguccugagagcucuaaa

SEQ ID NO: 16 - RNA, 19 nt, Artificial
  siRNA
gucucagccuuacgcaccu

SEQ ID NO: 17 - DNA, 180 bp, Homo sapiens
gtgtttaacctgattttcacctgaaatgactgatatagttttcagggcccggaggactcggaaggtgcgtaaggctgagacattgcccaggtccacaaattcagttgtgtatctgtaacaagggaaattcacacacgagacaatgacaacacaccaataggagacacacagtcagaggag

SEQ ID NO: 18 - DNA, 184 bp, Homo sapiens
gaaagggggtggggaagacagagagagagtcacttgtagctgagatctgagaggcaaacctgggcatcttacctgggataactgaaatagtttttagagctctcaggactctgaaagttcgaagagccgacaaattgcctagttttatattttctgatacatacctgcagaatcaaaccacagt

SEQ ID NO: 19 - DNA, 90 bp, Homo sapiens
tacacaactgaatttgtggacctgggcaatgtctcagccttacgcaccttccgagtcctccgggccctgaaaactatatcagtcatttca

SEQ ID NO: 20 - protein, 30 aa, Homo sapiens
YTTEFVDLGNVSALRTFRVLRALKTISVIS

SEQ ID NO: 21 - DNA, 90 bp, Homo sapiens
tatgtatcagaaaatataaaactaggcaatttgtcggctcttcgaactttcagagtcctgagagctctaaaaactatttcagttatccca

SEQ ID NO: 22 - protein, 30 aa, Homo sapiens
YVSENIKLGNLSALRTFRVLRALKTISVIP

SEQ ID NO: 23 - protein, 2016 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 24 - protein, 30 aa, Artificial
  Consensus sequence
  VARIANT (7)..(7): Xaa is K or D
YVTEFVXLGNVSALRTFRVLRALKTISVIP

(19) United States
(12) Patent Application Publication
(10) Pub. No.: US 2020/0123544 A1
(43) Pub. Date: Apr. 23, 2020

(54) GENE THERAPY TARGETING THE NEONATAL FORM OF NAV1.5 FOR TREATING CANCER

(71) Applicant: Celex Oncology Innovations Limited, Liverpool (GB)

(72) Inventor: Mustafa Bilgin Ali Djamgoz, London (GB)

(73) Assignee: Celex Oncology Innovations Limited, Liverpool (GB)

(21) Appl. No.: 16/658,663

(22) Filed: Oct. 21, 2019

(30) Foreign Application Priority Data
Oct. 22, 2018 (EP) 18201870.5

(51) Int. Cl. C12N 15/113; C12N 9/22; A61K 9/127

(52) U.S. Cl. CPC C12N 15/113; C12N 9/22; C12N 2800/80; C12N 2310/14; A61K 9/127

(57) ABSTRACT

The present invention provides oligomeric compounds, in particular oligonucleotide compounds, and methods of treating a cancer using such oligomeric compounds. Particularly contemplated are oligomeric compounds which comprise a target binding domain that is specifically hybridisable to mRNA or genomic DNA encoding neonatal Nav1.5. In cancer cells that express nNav1.5, the oligomeric compound can reduce the level of nNav1.5 mRNA in cancer cells, the level of nNav1.5 in the cancer cells and/or the level of nNav1.5 expressed on the surface of the cancer cells. This is turn can reduce or prevent metastatic behaviour of the cancer, pain sensation in the patient, invasiveness of the cancer and/or overall aggressiveness of the cancer.

### FIELD OF THE INVENTION

This invention relates to the treatment of cancer, and relates particularly to all cancers found to express a voltage-gated sodium channel (VGSC), such as, but not exclusively, treatment of colorectal cancer, breast cancer, lung cancer, ovarian cancer, astrocytoma and/or neuroblastoma.

### BACKGROUND OF THE INVENTION

The annual incidence of colorectal cancer (CRCa) is expected to increase globally by some 80% (to ˜2.2 million) over the next 20 years, with 62% of cases occurring in less developed countries (Karsa et al., 2010; Torre et al., 2016). There has also been a trend for CRCa to be diagnosed in younger people and these tend to be late-stage (e.g. You et al., 2012). This is a heterogeneous disease demonstrating varied genetic and epigenetic mechanisms (e.g. Ogino et al., 2011). Most CRCa cases are adenocarcinomas developing in a complex, multistep process known as the “adenoma-carcinoma sequence” (e.g. Fearon, 2011). Major problems remain in clinical management of CRCa, especially for patient subgroups that cannot be treated by surgery alone. This is mainly due to the absence of effective functional biomarkers of disease progression and eventual onset of chemoresistance during available therapies and metastasis (Van Emburgh et al., 2014). Consequently, novel predictive biomarkers and personalised treatment regimens are urgently needed.

It has been known for some time that several major human carcinomas express functional voltage-gated Na⁺ channels (VGSCs) which promote their cellular invasiveness in vitro and metastasis in vivo (Campbell et al., 2013; Driffort et al., 2014; Fraser et al., 2005; House et al., 2010; Laniado et al., 1997; Nelson et al., 2015; Roger et al., 2003; Yildirim et al., 2012). House et al. initially investigated this phenomenon in human CRCa and showed that the Nav1.5 subtype of VGSC occurred functionally in CRCa cell lines (House et al., 2010, 2015). In biopsies, also, Nav1.5 protein expression was upregulated. Importantly, computational analysis revealed SCN5A (the gene encoding Nav1.5) to be an upstream “key regulator” of CRCa invasiveness, driving a network of canonical genes including those for Ca²⁺ signalling, Wnt signalling, MAP kinase and proteases (House et al., 2010).

Nav1.5 is developmentally regulated via alternative splicing of exon 6, giving rise to ‘adult’ and ‘neonatal’ variants of the Nav1.5 protein that differ in the S3-S4 region of domain I by several amino acids (Fraser et al., 2005). This difference enabled a polyclonal antibody (NESOpAb) specific for the neonatal splice form of Nav1.5 (nNav1.5) to be produced (Chioni et al., 2005). In breast cancer, the functional VGSC was shown to be nNav1.5 (Brackenbury et al., 2007; Fraser et al., 2005). This is in line with the expression being ‘oncofetal’ (e.g. Ben-Porath et al., 2008). Originally, House et al. stated that the Nav1.5 in CRCa was the ‘adult’ form (aNav1.5) (House et al., 2010). Subsequently, nNav1.5 mRNA was shown to be expressed in vitro (Baptista-Hon et al., 2014).

A characteristic of growing tumours can be the development of internal hypoxia which may promote their metastatic potential (e.g. Krishnamachary et al., 2003), e.g., an increased invasiveness (e.g. Hongo et al., 2013). However, most work on control of cancer cell behaviour by ion channels has been done under normoxic conditions. In particular, the possible involvement of VGSC (nNav1.5) activity in the effects of hypoxia is not known.

Accordingly, despite the progress in the art, there is still a need for new methods and compounds for treating cancer, particularly cancers associated with nNav1.5 expression. It is an object of the present invention to provide such methods and compounds.

### SUMMARY OF THE INVENTION

It has been found by the present inventor(s) that gene therapy targeting the nNav1.5 gene or mRNA can be used for treatment of cancer.

Accordingly, the present invention relates to methods of treating cancer comprising administering an oligomeric compound comprising a target binding domain that is specifically hybridisable to mRNA or genomic DNA encoding Nav1.5, such as nNav1.5.

The invention also relates to oligomeric compounds, in particular oligonucleotide compounds, useful for such methods.

These and other aspects and embodiments provided for by the present invention are illustrated below.

### FIGURE LEGENDS

FIG. 1. Neonatal Nav1.5 (nNav1.5) mRNA and protein expression in human CRCa cell lines (HT29, HCT116 and SW620). (A) mRNA expression levels of neonatal SCN5A, normalised to SDHA by the 2^{×ΔΔC(t)} method, and plotted relative to the level in HT29 cells. Each histobar indicates mean±SE (n=6). (**) denotes P<0.01. (B) Western blots carried out on total protein (50 μg) from the same panel of CRCa cell lines, using the nNav1.5-specific NESOpAb antibody. Upper bands indicate protein of the expected size (220 kDa). α-actinin was used for loading control (lower bands). (C) The levels of nNav1.5 protein expression in the 3 cell lines. Data (mean±SE; n=5) are plotted relative to the level in HT29 cells. Statistics: (***)=P<0.001 for HT29 vs. HCT116; (*)=P<0.005 for HCT116 vs. SW620. p=0.058 for HT29 vs. SW620 (not indicated). (D) Immunocytochemical staining of HT29, HCT116 and SW620 cells using the NESOpAb antibody at 1:100 dilution of a 0.7 mg/ml stock. Cells were not permeabilized. Both immunofluorescence and matching phase contrast images are shown. Scale bar (30 μm) applies to all panels. (E) Immunocytochemistry data quantified as immunofluorescence intensity normalized to cell area. (F) Percentage of cells stained. Error bars represents SEs (n=350 cells for each cell; 3 independent biological repeats). Statistical significance was determined relative to HT29 cells: (**) and (***) denote P<0.01 and P<0.001, respectively.

FIG. 2. Comparison of electrophysiological effects of treating SW620 cells with siRNAs targeting either nNav1.5 (with n3-siRNA) or aNav1.5 (with a-siRNA). Square symbols, n3-siRNA treatment; circles, a-siRNA treatment (key applies to all parts of figure). (A) Current-voltage relationships. Inset shows normalized representative current traces obtained following treatment with either n3-siRNA or a-siRNA. (B) Conductance-voltage relationships. (C) Steady-state inactivation (“availability”). (D) Recovery from inactivation (It/Ic). For all the data, recordings were from cells following 96 h of transfection and 24 h of serum starvation. Each data point denotes mean±SE (n=5-9 cells). (All symbols are defined in the text.) Statistical significance between individual n3-siRNA or aNav1.5 data points are given by *=P<0.05; **=P<0.01; ***=P<0.01.

FIG. 3. Effects targeting neonatal or adult Nav1.5 with specific siRNAs on Matrigel invasion of SW620 cells with and without TTX co-treatment. Cells were serum-starved for 24 h prior to treatments and then allowed to invade over 48 h. Invasiveness is presented as ‘box plots’ relative to the largest value observed between treatment conditions for each individual experiment. (A) Data from cells treated with (i) control siRNA (c-siRNA); (ii) three different siRNAs targeting nNav1.5 (n1,n2,n3-siRNA); and siRNA targeting ‘adult’ Nav1.5 (a-siRNA). (B) Similar to (A), cells were treated with control siRNA (c-siRNA); control siRNA+20 μM TTX (c+TTX); siRNA targeting nNav1.5 (n3-siRNA); n3-siRNA with 20 μM TTX (n3+TTX); siRNA targeting ‘adult’ Nav1.5 (a-siRNA); and a-siRNA+(20 μM TTX (a+TTX). Statistical significance: (x)=P>0.05. (*)=P<0.05. (**)=P<0.01. (***)=P <0.001). Each experiment was performed 4-8 times.

FIG. 4. Effect of hypoxia on SW620 cell invasiveness. Cells were serum-starved for 24 h prior to treatments and then allowed to invade over 48 h. Invasiveness is presented as ‘box plots’ relative to the largest value observed between treatment conditions for each individual repeat. Data are presented as means±SE (n=5). Normoxia (white box); hypoxia (grey box). Hypoxia (1% O₂; 72 h) caused a significant increase in invasiveness compared to the normoxia control. The hypoxia-induced increase in invasiveness was completely suppressed following treatment of the cells with siRNA targeting nNav1.5. The control siRNA (“si-Control”) had no effect on invasiveness. Statistical significance: (**)=P<0.01. (***)=P<0.001.

FIG. 5. Ranolazine reduced the hypoxia-induced increase in invasiveness in SW620 cells through nNav1.5. (A) SW620 cell invasion (over 72 h) was significantly reduced by 20 μM TTX and by 5 μM ranolazine, compared with the respective controls. Data are presented as means±SE (n=5). Normoxia (white box); hypoxia (grey box). (B) Dose-dependent inhibition of c-siRNA-treated SW620 cell invasion (over 72 h under 1% O₂) by ranolazine (1, 5 and 10 μM) (closed symbols/solid line). The effect of ranolazine was lost in cells transfected with n3-siRNA (open symbols/dotted line). +1 μM ranolazine treated cells showed significant reduction in invasiveness compared to c-siRNA+1 μM ranolazine treated cells. Niether n3-siRNA+5 μM ranolazine nor n3-siRNA+10 μM ranolazine treated cells showed significant reduction in invasiveness compared to the respective controls. Data are presented as means±SEs (n=3). Statistical significance: (**)=P<0.01. (***)=P<0.001.

FIG. 6. Effects of VGSC inhibition on nNav1.5 mRNA and protein expression in SW620 cells under normoxia and hypoxia. (A) Lack of effect of TTX (20 μM) and ranolazine (5 μM) on nNav1.5 mRNA expression in normoxia. Hypoxia (1% O₂ for 48 h) significantly increased nNav1.5 mRNA expression. This was inhibited by the same treatments with TTX and ranolazine, as above. Data are presented as means±SE (n=5). Normoxia (white box); hypoxia (grey box). Statistical significance: (***)=P<0.001). (B) Lack of effect of hypoxia and co-treatment with TTX (20 μM) or ranolazine (5 μM) on nNav1.5 protein expression. Typical western blots are shown, using the nNav1.5-specific NESO-pAb antibody (˜220 kDa) and α-actinin as loading control (˜100 kDa). Quantitative data are presented as means±SEs (n=4). Normoxia (white box); hypoxia (grey box).

FIG. 7. Reduction of voltage-gated Na− channel (nNavL5) expression with time in culture. (A) Box plots illustrating effects of time in culture (24 vs. 72 h) on voltage-gated Na− channel peak current density (pA/pF) in SW620 cells. Recordings were from n=30 and 17 individual cells, respectively. ***=P<0.001. (B) Immunocytochemical staining of human SW620 cells using a nNav1.5-specific voltage gated Na-channel antibody (NESOpAb) at 1:100 dilution of a 0.7 mg/ml stock Cells were non-permeabilized. Images are either phase contrast (PC) or fluorescence (FL) for either 24 h (top) or 72 h (bottom) in culture. Scale bar applies to all panels.

FIG. 8. Optimization of Matrigel concentration on invasiveness of SW620 cells. Histogram summarizing the effect of Matrigel concentration on invasiveness of SW620 cells. Inserts were coated with 50 μl of varying concentrations of Matrigel as indicated. Cells were serum-starved for 24 hours prior to plating on inserts and invasion occurred over 48 hours. The density of invaded cells was determined by averaging cell counts from 12 randomly chosen fields of view for each insert.

FIG. 9. Specificity of the NESOpAb antibody. Representative immunoblot showing specifity of NESOpAb antibody for neonatal Nav1.5 (nNav1.5). This is demonstrated using EBNA-293 cell line transfectants including empty plasmid (control—EBNA-C); ‘neonatal’ Nav1.5 cDNA plasmid (positive control—EBNA-N) and ‘adult’ Nav1.5 cDNA plasmid (negative control—EBNA-A), respectively. Tue immunoblot is typical of three independent repeats.

FIG. 10. Effects of siRNAs targeting either nNav1.5 or aNav1.5 on the functional characteristics (kinetics) of voltage-gated Na− channels in SW620 cells. Electrophysiological effects of treating SW620 cells with siRNAs targeting either nNav1.5 with n3-siRNA (A-C) or aNav1.5 with a-siRNA (D-F). Recordings were from cells following a 96 h transfection and 24 h serum starvation. Square symbols, controls; circles, si-RNA treatments (insets in A and D apply to all other parts). Each data point denotes mean±SEM (n=4-13 cells). (A & D) Current-voltage relationships. (B & E) Steady-state inactivation (“availability”). (C & F) Recovery from inactivation (Iₜ/I_{c}).

FIG. 11. Comparison of the time-dependent effects of normoxia and hypoxia (1% O₂) on growth of the SW620 cells. (A) Cell growth over 120 h as determined by the MTT assay of proliferation. Values after 48 h are significantly different between normoxic and hypoxic conditions (P<0.05, for 72 h and 96 h; P<0.001 for 120 h). (B) Cell growth over 120 h as determined by the direct counting of cells. Values after 48 h are significantly different between normoxic and hypoxic conditions (P<0.05, for 72 h and 96 h ; P<0.01 for 120 h).

FIG. 12. Nav 1.5 splice variant sequences encoded by exon 6 of the SCN5A gene and a consensus sequence (SEQ ID NO:24).

FIG. 13. Nucleotide and amino acid sequences of nNav1.5 and aNav1.5 in the splicing region of domain 1/exon 6. A) Chromosomal DNA segments, where the segment corresponding to exon 6 for adult Nav1.5 underlined in SEQ ID NO:17 and the segment corresponding to exon 6 for neonatal Nav1.5 is underlined in SEQ ID NO:18. B) Top: The cDNA (SEQ ID NO:19) and amino acid (SEQ ID NO:20) sequence corresponding to exon 6 of adult Nav1.5 (aNav1.5). Bottom: The cDNA (SEQ ID NO:21) and amino acid (SEQ ID NO:22) sequence corresponding to exon 6 of neonatal Nav1.5 (nNav1.5).

### DETAILED DISCLOSURE OF THE INVENTION

Functional expression of voltage-gated Na+channels (VGSCs) occurs in human carcinomas and promotes invasiveness in vitro and metastasis in vivo (Table 1). Both neonatal and adult forms of Nav1.5 (nNav1.5 and aNav1.5, respectively) have been reported to be expressed at mRNA level in colorectal cancer (CRCa) cells.

TABLE 1
VGSC subtype Nav1.5 expression in human carcinomas

| Carcinoma | VGSC subtype(s) | Reference(s) |
|---|---|---|
| Breast | nNav1.5 | Fraser et al., 2005; Martin and |
| | (and nNav1.7) | Zukin, 2006; Nelson et al., |
| | | 2015: Driffort et al., 2014 |
| Colon | nNav1.5 | Guzel et al., 2019; House et al., |
| | | 2010; Baptista-Hon et al., 2014 |
| Ovary | Nav1.5 | Gao et al., 2010; Gao et al., 2019 |
| Melanoma | Nav1.5 | Xie et al., 2018 |
| Oral squamous | Nav1.5 | Zhang et al., 2019 |
| cell carcinoma | | |
| Astrocytoma | nNav1.5 | Xing et al., 2014 |
| Neuroblastoma | nNav1.5 | Ou et al., 2005 |

As reported in Example 1, three CRCa cell lines (HT29, HCT116 and SW620) were studied and found to express nNav1.5 mRNA and protein. In SW620 cells, adopted as a model, effects of gene silencing (by several siRNAs) selectively targeting nNav1.5 or aNav1.5 were determined on (i) channel activity and (ii) invasiveness in vitro. Silencing nNav1.5 made the currents more ‘adult-like’ and suppressed invasion by up to 73%. Importantly, subsequent application of the highly specific, general VGSC blocker, tetrodotoxin (TTX), had no further effect. Conversely, silencing aNav1.5 made the currents more ‘neonatal-like’ but suppressed invasion by only 17% and TTX still induced a significant effect. Hypoxia increased invasiveness and this was also blocked completely by siRNA targeting nNav1.5. The effect of hypoxia was suppressed dose-dependently by ranolazine but its effect was lost in cells pre-treated with nNav1.5-siRNA. Based on these results, it could be concluded (i) that functional nNav1.5 expression was common to human CRCa cells; (ii) that hypoxia increased the invasiveness of the cells tested; (iii) that the VGSC-dependent invasiveness was driven predominantly by nNav1.5 under both normoxic and hypoxic conditions; and (iv) that the hypoxia-induced increase in invasiveness was likely to be mediated by the persistent current component of nNav1.5.

Definitions

Before the present invention is described in greater detail, it is to be understood that this invention is not limited to particular aspects and embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs.

“Voltage-gated sodium channels” or “VGSCs” is a known class of integral membrane proteins that form ion channels, conducting sodium ions (Na+) through a cell's plasma membrane. In humans, there are nine genes (SCN1A, SCN2A, SCN3A, SCN4A, SCN5A, SCN8A, SCN9A, SCN10A, and SCN11A) which encode for nine different VGSC alpha subunit or “Nav” proteins (Nav1.1 to Nav1.9, respectively).

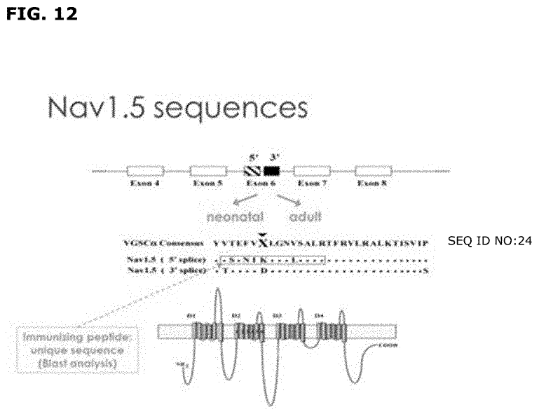

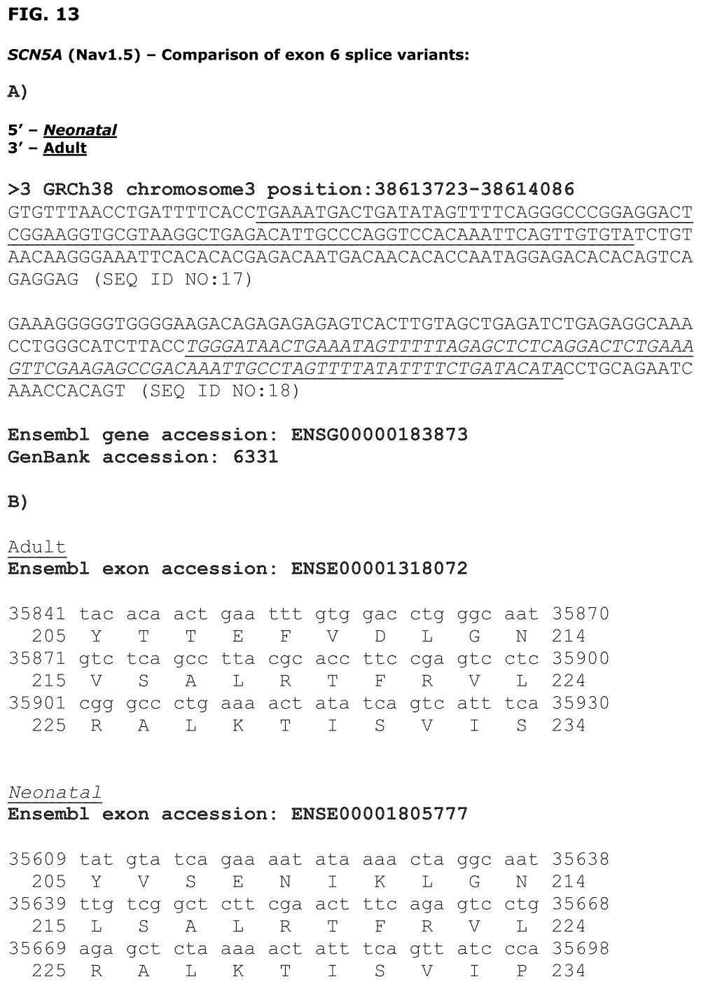

As used herein, “Nav1.5” refers to the human Nav1.5 protein (in adult or neonatal form) encoded by the gene SCNSA (Genbank Gene ID: 6331), a highly conserved gene located on human chromosome 3, where it spans more than 100 kb. The gene consists of 28 exons, of which exon 1 and part of exon 2 form the 5′ untranslated region (5′UTR) and exon 28 forms the 3′ untranslated region (3′UTR) of the RNA. More than 10 different splice isoforms have been described for SCN5A, of which several harbour different functional properties. Furthermore, different isoforms are expressed during fetal and adult life, providing for differences in exon 6. The fetal and adult form of the protein have amino acid differences in, e.g., the DI:S3-S4 region of the channel protein (see, e.g., FIGS. 12 and 13).

As used herein, “neonatal Nav1.5” (nNav1.5), also referred to herein as “fetal Nav1.5”, comprises an amino acid sequence that differs from SEQ ID NO:1 (UniProtKB—Q14524 (SCN5A_HUMAN); Isoform 1, as accessed on 22 Oct. 2018), in at least amino acid residue 211, such as in amino acid residues 206, 207, 209, 210, 211, 215, and 234. Preferably, in nNav1.5, the amino acid at position 211 of SEQ ID NO:1 is K (Lys). For example, the nNav1.5 may comprise amino acid residues V, S, N, I, K, L, and P in positions 206, 207, 209, 210, 211, 215, and 234 respectively, where aNav1.5 comprises the amino acid residues T, T, F, V, D, V, and S in the same/corresponding positions. In one embodiment, in the neonatal variant, residues 206-211 of SEQ ID NO:1 are changed from TTEFVD→VSENIK, optionally wherein, in the neonatal variant, the amino acid residue at position 215 is changed from V→L and/or the amino acid residue at position 234 is changed from S→P. Specific examples of nNav1.5 amino acid sequences are illustrated in FIGS. 12 and 13 as well as in SEQ ID NO:23 (UniProtKB—H9KVD2 (H9KVD2_HUMAN)). Examples of mRNA sequences for aNav1.5 and nNav1.5 are shown herein in SEQ ID NOS:2 and 3, respectively, and mRNA sequences transcribed from exon 6 in aNav1.5 and nNav1.5 and encoding amino acids 205 to 234 of the respective Nav1.5 protein are shown in SEQ ID NO:19 and 21, respectively. As is typical in a bioinformatics context, however, these sequences are expressed as cDNA bases (GCAT) rather than RNA bases (GCAU).

The percent identity between two sequences is a function of the number of identical positions shared by the sequences (i.e., % homology=# of identical positions/total # of positions×100), taking into account the number of gaps, and the length of each gap, necessary for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444 453 (1970) algorithm.

As used herein, a sequence that is “similar” to a reference sequence typically has a sequence identity of at least about 80%, such as at least about 85%, such as at least about 90%, such as at least about 95%, such as at least about 96%, 97%, 98% or 99% to the reference sequence; whereas a sequence “identical” to a reference sequence has a sequence identify of 100% to the reference sequence. Further, unless otherwise indicated or contradicted by context, when referring herein to sequence similarity or identity between an mRNA sequence and a DNA sequence, it is to be understood that the similarity or identity is between the two sequences in the same format, i.e., either DNA (CGAT) or RNA (CGAU), so that all thymine (T) are exchanged for uracil (U) in the DNA sequence or vice versa in the RNA sequence.

So, for example, in one embodiment, the mRNA encoding Nav1.5 comprises a sequence similar or identical to SEQ ID NO:2 (NCBI Reference Sequence: NM_198056.2; accessed on 22 Oct. 2018; encoding SEQ ID NO:1) or SEQ ID NO:3 (NCBI: NM_001099404.1; accessed 5 Oct. 2019).

As used herein, “target binding domain” refers to a domain of an oligomeric compound (or even the oligomeric compound as such) which binds to a specified target sequence, which according to the present invention is mRNA or genomic DNA encoding human Nav1.5 (in adult and/or neonatal form).

As used herein, “hybridisation” means hydrogen bonding, which may be Watson-Crick, Hoogsteen, reversed Hoogsteen hydrogen bonding, etc. between complementary nucleoside or nucleotide bases. Watson and Crick showed approximately fifty years ago that deoxyribo nucleic acid (DNA) is composed of two strands which are held together in a helical configuration by hydrogen bonds formed between opposing complementary nucleobases in the two strands. The four nucleobases, commonly found in DNA are guanine (G), adenine (A), thymine (T) and cytosine (C) of which the G nucleobase pairs with C, and the A nucleobase pairs with T. In RNA, the nucleobase thymine is replaced by the nucleobase uracil (U), which similarly to the T nucleobase pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

The term “specifically hybridisable” means that the target binding domain, optionally the oligomeric compound, in question is capable of binding sufficiently strong and specific to the target mRNA or genomic DNA to provide the desired interference with the normal function of the target mRNA or DNA whilst leaving the function of non-target mRNAs or DNAs unaffected. The relevant hybridisation and thereby interference with the function normally takes place at physiological conditions, i.e. at about 37° C. In vitro conditions for evaluating specific hybridization can, for example, be stringent conditions, where stringent conditions can include: 400 mM NaCl, 40 mM PIPES pH 6.4, 1 mM EDTA, 50 degrees centigrade or 70 degrees centigrade for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of specific hybridization of two sequences in accordance with the ultimate application of the hybridised nucleotides. Specific hybridization does, however, not exclude that one or two mismatches can be present in the target binding domain, e.g., at its 5′ and/or 3′ terminals. Preferably, the target binding domain includes no mismatches or at the most one mismatch with the target sequence. In this context, “directly complementary” means that any “G” in the target binding domain pairs with a “C” in the mRNA or genomic DNA sequence and that any “A” in the target binding domain pairs with a “U” in the mRNA sequence or with a “T” in the genomic DNA sequence.

As used herein, in the context of an oligomeric compound in double-stranded form (e.g., a duplex siRNA or dsRNA), the term “antisense strand” or “guide strand” refers to the strand that comprises a target binding domain specifically hybridisable to a target sequence in an mRNA or DNA sequence. The term “sense strand” refers to the second strand, which comprises a domain that is substantially or directly complementary to a segment of the antisense strand, resulting in the double stranded form.

Aspects and Embodiments

Certain aspects and embodiments according to the invention are set forth below.

In one aspect, the invention provides a method of treating a cancer comprising cancer cells that express the neonatal form of human Nav1.5 (nNav1.5), comprising administering to a subject suffering from said cancer an oligomeric compound comprising a target binding domain that is specifically hybridisable to mRNA or genomic DNA encoding nNav1.5, wherein the oligomeric compound reduces the level of mRNA encoding nNav1.5 in the cancer cells, the level of nNav1.5 in the cancer cells and/or the level of nNav1.5 expressed on the surface of the cancer cells. In some embodiments, the cancer is colorectal cancer, breast cancer, lung cancer, ovarian cancer, astrocytoma or neuroblastoma, or a combination of any thereof. In a specific embodiment, the cancer is colorectal cancer.

In one aspect, the invention provides a method of treating a cancer selected from colorectal cancer, breast cancer, lung cancer, ovarian cancer or neuroblastoma, or a combination of any thereof, wherein the oligomeric compound comprises a target binding domain that is specifically hybridisable to messenger RNA (mRNA) or genomic DNA encoding nNav1.5.

In some embodiments, the target binding domain is specifically hybridisable to mRNA encoding nNav1.5. In one embodiment, the nNav1.5 comprises a Lys (K) in position 211 of SEQ ID NO:1. The nNav1.5 may, for example, comprise the amino acids V, S, N, I, K, L, and P in positions 206, 207, 209, 210, 211, 215, and 234 of SEQ ID NO:1, respectively.

In some embodiments, the mRNA comprises a segment at least about 90%, such as at least about 95%, such as at least about 96%, 97%, 98%, 99% or 100% identical to a sequence directly complementary to SEQ ID NO:21.

In some embodiments, the target binding domain, the oligomeric compound, or both, is a 15, 16, 17, 18, 19, 20, 21, 22, 23, 24, 25, 26, 27, 28, 29 or 30-mer, optionally in double-stranded form. The target binding domain, the oligomeric compound, or both, may, for example, be a ribonucleic acid (RNA), deoxyribonucleic acid (DNA), peptide nucleic acid (PNA), locked nucleic acid (LNA), unlocked nucleic acid (UNA), a phosphorodiamidate Morpholino oligomer (PMO) molecule, or a combination of any two or more thereof.

In some embodiments, the oligomeric compound is comprised in or encoded by a vector, such as a viral vector, optionally wherein the vector further comprises one or more expression control sequences. In one embodiment, the vector may further comprise a transactivating crRNA (tracrRNA), a nucleic acid encoding a CRISPR-associated enzyme selected from Cas9 and Cpf1, or both.

In some embodiments, the target binding domain, the oligomeric compound, or both, is an RNA molecule selected from an small interfering RNA (siRNA), short hairpin RNA (shRNA), a guide RNA (gRNA), single guide RNA (sgRNA), or CRISPR RNA (crRNA) molecule. In a specific embodiment, the oligomeric compound is an siRNA molecule, optionally in double-stranded form.

In some embodiments, the target-binding domain is specifically hybridisable or directly complementary to a contiguous portion of residues 797 to 896 of SEQ ID NO:3.

In some embodiments, the target binding domain is specifically hybridisable or directly complementary to genomic DNA transcribed into GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or to two or all of SEQ ID NOS:13-15, or to mRNA transcribed therefrom.

In some embodiments, the oligomeric compound comprises or consists of the RNA sequence (in 5′→3′ direction) GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or a combination of two or all thereof, optionally in double-stranded form.

In some embodiments, the oligomeric compound is comprised in a lipid nanoparticle (LNP) or liposome.

In some embodiments, the method reduces or prevents metastatic behaviour of the cancer, pain sensation in the subject, invasiveness of the cancer, overall aggressiveness of the cancer, or any combination thereof. In some embodiments, the method comprises determining that the cancer expresses nNav1.5 prior to administering the oligomeric compound. In some embodiments, the cancer comprises one or more hypoxic tumours. In some embodiments, the method comprises administering a second therapeutic agent to the subject. In one embodiment, the second therapeutic agent is not a VGSC blocker.

In a third aspect, the invention provides an isolated oligomeric compound comprising or consisting of the RNA sequence GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or an RNA sequence directly complementary to SEQ ID NO:15, SEQ ID NO:13 or SEQ ID NO:14 in double-stranded form with a complementary RNA sequence.

The following particular aspects and embodiments are also contemplated:

In one aspect, the invention provides an oligomeric compound for use in treating or preventing cancer comprising cancer cells that express the neonatal form of human Nav1.5 (nNav1.5), wherein the oligomeric compound comprises a target binding domain that is specifically hybridisable to messenger RNA (mRNA) or genomic DNA encoding Nav1.5, and reduces the level of neonatal Nav1.5 (nNav1.5) mRNA in the cancer cells, the level of nNav1.5 expressed on the surface of the cancer cells, or both. In some embodiments, the cancer is colorectal cancer, breast cancer, lung cancer, ovarian cancer or neuroblastoma, or a combination of any thereof. In some embodiments, the target binding domain is specifically hybridisable to messenger RNA (mRNA) or genomic DNA encoding nNav1.5.

In one aspect, the invention provides an oligomeric compound for use in treating or preventing a cancer selected from colorectal cancer, breast cancer, lung cancer, ovarian cancer or neuroblastoma, or a combination of any thereof, wherein the oligomeric compound comprises a target binding domain that is specifically hybridisable to messenger RNA (mRNA) or genomic DNA encoding nNav1.5.

In some embodiments, the cancer is colorectal cancer. In some embodiments, the nNav1.5 comprises a Lys (K) in position 211 of SEQ ID NO:1, optionally wherein the nNav1.5 comprises the amino acids V, S, N, I, K, L, and P in positions 206, 207, 209, 210, 211, 215, and 234 respectively. In some embodiments, the mRNA is at least about 90%, such as at least about 95%, such as at least about 96%, 97%, 98%, 99% or 100% identical to SEQ ID NO:2 identical to a sequence directly complementary to SEQ ID NO:2 (encoding SEQ ID NO:1). In some embodiments, the target binding domain is a ribonucleic acid (RNA), deoxyribonucleic acid (DNA), peptide nucleic acid (PNA), locked nucleic acid (LNA), unlocked nucleic acid (UNA), a phosphorodiamidate Morpholino oligomer (PMO) molecule, or a combination of any two or more thereof. In some embodiments, the oligomeric compound is an RNA molecule selected from an small interfering RNA (siRNA), short hairpin RNA (shRNA), a guide RNA (gRNA), single guide RNA (sgRNA), or CRISPR RNA (crRNA) molecule. In some embodiments, the oligomeric compound is comprised in a vector, such as a viral vector, optionally wherein the vector further comprises one or more expression control sequences. In some embodiments, the vector further comprises a transactivating crRNA (tracrRNA), a nucleic acid encoding a CRISPR-associated enzyme selected from Cas9, or both. In some embodiments, the oligomeric compound is a siRNA molecule, optionally in double-stranded form. In some embodiments, the oligomeric compound or vector is comprised in a lipid nanoparticle (LNP). In some embodiments, the target binding domain is specifically hybridisable to genomic DNA transcribed into GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or to two or all of SEQ ID NOS:1-3, or to mRNA transcribed therefrom. In some embodiments, the target binding domain is a 15, 16, 17, 18, 19, 20, 21, 22, 23, 24, 25, 26, 27, 28, 29 or 30-mer. In some embodiments, the oligomeric compound is a 15, 16, 17, 18, 19, 20, 21, 22, 23, 24, 25, 26, 27, 28, 29 or 30-mer, optionally in double-stranded form. In some embodiments, the oligomeric compound comprises or consists of the RNA sequence (in 5′→3′ direction) GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or a combination of two or all thereof, optionally in double-stranded form. In some embodiments, the use reduces or prevents metastatic behaviour of the cancer and/or pain sensation in a patient suffering from the cancer. In some embodiments, the use comprises determining that the cancer expresses nNav1.5 prior to the use. In some embodiments, the use according to any one of the preceding embodiments is in combination with a second therapeutic agent. In some embodiments, said second therapeutic agent is ranolazine or eleclazine. Suitable dosages of ranolazine and eleclazine can be found in WO 2018/146313 (Celex GmbH) and WO2012/049440 (Celex Oncology Ltd.), both of which are hereby incorporated by reference in their entireties. In some embodiments, the cancer comprises a hypoxic tumor.

In one aspect, the invention provides an isolated oligomeric compound comprising or consisting of the RNA sequence GAGUCCUGAGAGCUCUAAA (NESO; SEQ ID NO:15); CUAGGCAAUUUGUCGGCUC (Neo1; SEQ ID NO:13) UAUCAUGGCGUAUGUAUCA (Neo2; SEQ ID NO:14), or of SEQ ID NO:1, 2 or 3, optionally in double-stranded form with a complementary RNA sequence.

In one aspect, the invention method of treating a cancer comprising cancer cells that express the neonatal form of human Nav1.5 (nNav1.5), comprising administering to a subject suffering from said cancer an oligomeric compound comprising a target binding domain that is specifically hybridisable to messenger RNA (mRNA) or genomic DNA encoding Nav1.5, wherein the oligomeric compound reduces the level of nNav1.5 mRNA in the cancer cells, the level of nNav1.5 expressed on the surface of the cancer cells, or both. In some embodiments, the method further comprises the features of any one or more of the above-mentioned embodiments.

In one aspect, the invention provides a method of treating a cancer selected from colorectal cancer, breast cancer, lung cancer, ovarian cancer or neuroblastoma, or a combination of any thereof, wherein the oligomeric compound comprises a target binding domain that is specifically hybridisable to messenger RNA (mRNA) or genomic DNA encoding nNav1.5. In some embodiments, the method further comprises the features of any one or more of the above-mentioned embodiments.

Oligomeric Compounds

The oligomeric compounds of the invention selectively inhibit the transcription, translation and/or expression one or more Nav1.5 proteins of interest, such as nNav1.5.

Target-Binding Domain

Oligomeric compounds of the present invention comprise, and may in some embodiments even consist of, a target-binding domain that binds to a specified target sequence, which is typically mRNA or genomic DNA encoding human Nav1.5 (in adult and/or neonatal form). A skilled person can adapt the length of the oligomeric compound to a suitable length for the intended application. For some applications, however, the target binding domain and/or the oligomeric compound may be a 15, 16, 17, 18, 19, 20, 21, 22, 23, 24, 25, 26, 27, 28, 29 or 30-mer. Accordingly, target binding domains of 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 mer-units in length are contemplated. The oligomeric compound may optionally be in double-stranded form. A double-stranded form usually includes an antisense and a sense strand, where the antisense strand comprises or consists of the target-binding domain and the sense strand is complementary, preferably directly complementary, to a sufficient portion of the antisense strand so as to form a duplex.

The target binding domain is specifically hybridisable to a target sequence in the mRNA or genomic DNA sequence encoding Nav1.5 (in adult and/or neonatal form). Preferably, the target binding domain is at least specifically hybridisable to a target sequence in the mRNA or genomic DNA encoding the neonatal form, i.e., nNav1.5. Accordingly, in some embodiments, the target binding domain can specifically hybridise to a target sequence common to the nNav1.5 and aNav1.5 mRNA, or common to the nNav1.5 and aNav1.5 genomic DNA.

In preferred embodiments, however, the target sequence exists only in the mRNA or genomic DNA sequence encoding nNav1.5. In such instances, the target sequence can be fully or partially located in exon 6 of the genomic Nav1.5 DNA or in the mRNA segment transcribed therefrom. The target binding domain is thereby specifically hybridisable to the mRNA or genomic DNA sequence encoding nNav1.5, but less hybridisable, or even unable to hybridise, to the mRNA or genomic DNA sequence encoding aNav1.5. Representative genomic and cDNA (i.e., mRNA presented in DNA format) sequences corresponding to the adult and neonatal versions of exon 6 are shown in FIG. 13.

In some embodiments, the target binding domain is directly complementary to a target sequence in mRNA or genomic DNA encoding Nav1.5 in adult or neonatal form. In a particular embodiment, the target binding domain is directly complementary to a target sequence in mRNA or genomic DNA encoding neonatal Nav1.5 (nNav1.5). In another particular embodiment, the target binding domain is directly complementary to a target sequence in mRNA or genomic DNA encoding neonatal Nav1.5, but not to mRNA or genomic DNA encoding adult Nav1.5 (aNav1.5).

Target Sequence

The target sequence is typically a contiguous portion of the mRNA or genomic DNA and is at least long enough to serve as a binding partner (typically hybridization partner) for the target-binding domain. The target sequence may, for example, comprise or consist of 15, 16, 17, 18, 19, 20, 21, 22, 23, 24, 25, 26, 27, 28, 29 or 30 or more contiguous nucleobases. Accordingly, target sequences of 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 contiguous nucleotides in length are contemplated.

Preferably, an mRNA target sequence is located within the protein coding region, and a genomic DNA target sequence located at least partially within an exon. The mRNA can be a primary transcription product, such as a precursor mRNA sequence, or mature mRNA resulting from processing of the primary transcription product, such as splicing.

In some embodiments, the target sequence is a contiguous portion of the nucleotide sequence of an mRNA molecule resulting from the transcription of the Nav1.5 gene SCNSA (in adult and/or neonatal form). For example, the target sequence may exist in both aNav1.5 and nNav1.5. In such embodiments, the target sequence may be similar or identical to a contiguous portion of SEQ ID NO:2 and SEQ ID NO:3 (in mRNA format).

In preferred embodiments, the target sequence is a contiguous portion of the nucleotide sequence of an mRNA molecule encoding Nav1.5 in neonatal form, i.e., nNav1.5. Preferably, the target sequence does not exist in aNav1.5 mRNA. In such embodiments, the target sequence may be similar or identical to a contiguous portion of SEQ ID NO:3 (in mRNA form), preferably a portion which is at least partially located in SEQ ID NO:21. Preferably, the target sequence is located within a Nav1.5 mRNA segment corresponding to residues 750 to 950, such as residues 790 to 900, such as residues 796 to 897 of said nNav1.5 mRNA, as represented by SEQ ID NO:3. In a preferred embodiment, the mRNA target sequence is fully or partially located within SEQ ID NO:21 and does not exist in SEQ ID NO:2.

In such embodiments, the target binding domain may be specifically hybridisable or directly complementary to a contiguous portion of SEQ ID NO:3 (in mRNA form), preferably a portion which is at least partially located in SEQ ID NO:21. Preferably, the target binding domain may be specifically hybridisable or directly complementary to a Nav1.5 mRNA segment corresponding to residues 750 to 950, such as residues 790 to 900, such as residues 796 to 897 of said nNav1.5 mRNA, as represented by SEQ ID NO:3 (in mRNA form), and does not specifically hybridize to SEQ ID NO:2 (in mRNA form). In a preferred embodiment, the target binding domain is specifically hybridisable or directly complementary to a contiguous portion of SEQ ID NO:21 (in mRNA form) and does not specifically hybridize to SEQ ID NO:2 (in mRNA form).

Preferred are target binding domains specifically hybridisable to genomic DNA transcribed into GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or to two or all of SEQ ID NOS:13-15, or to mRNA transcribed from such genomic DNA. The target binding domain may bind to either the coding strand or the non-coding strand of the genomic DNA. In one embodiment, the target binding domain binds to the non-coding strand.

Particularly preferred are target binding domains directly complementary to genomic DNA transcribed into GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or to two or all of SEQ ID NOS:13-15, or to mRNA transcribed from such genomic DNA.

In some embodiments, the target binding domain comprises or consist of a sequence directly complementary to one or more of SEQ ID NOS:13-15.

In some embodiments, the target binding domain comprises or consists of one or more of SEQ ID NOS:13-15.

In some embodiments, the target sequence is a contiguous portion of the nucleotide sequence of the Nav1.5 gene (in adult and/or neonatal form). Preferably, the target sequence exists in a nNav1.5 gene but not in aNav1.5 gene. In such embodiments, the target sequence may include at least a portion of the neonatal variant of exon 6 (see FIG. 13).

In some embodiments, the target binding domains may be directly complementary to all or part of SEQ ID NO:18, particularly to all or part of the segment in SEQ ID NO:18 that is underlined in FIG. 13. In some embodiments, the target binding domain is directly complementary to a sequence complementary to all or part of SEQ ID NO:18, particularly to all or part of the segment in SEQ ID NO:18 that is underlined in FIG. 13.

In some embodiments, the target binding domains may be directly complementary to all or part of SEQ ID NO:21. In some embodiments, the target binding domain is directly complementary to a sequence complementary to all or part of SEQ ID NO:21.

Kinds of Oligomeric Compounds

Many kinds of oligomeric compounds suitable for application in accordance with the present invention are known in the art. Oligonucleotide formats are particularly suitable, and include ribonucleic acid (RNA), deoxyribonucleic acid (DNA), peptide nucleic acid (PNA), locked nucleic acid (LNA), unlocked nucleic acid (UNA), a phosphorodiamidate Morpholino oligomer (PMO) molecule, and combinations of different forms of these formats in a single oligomeric compound. A preferred oligomeric compound is an siRNA molecule, in single- or double-stranded form (i.e., a duplex).

In some embodiments, the oligomeric compound comprises one or more nucleotide analogues, e.g., where the ribose ring is modified. An oligomeric compound can thus be prepared from (only) nucleotide analogues to form the desired oligonucleotide sequence, or nucleotide analogues can replace one or more nucleotides in a DNA or RNA sequence to form an oligomeric compound.

In one embodiment, the oligomeric compound comprises one or more LNAs (locked nucleic acids), often referred to as inaccessible RNA. An LNA is an RNA nucleotide analogue in which the ribose moiety is modified with an extra bridge connecting the 2′ oxygen and 4′ carbon. The bridge “locks” the ribose in the 3′-endo conformation, which is often found in A-form duplexes. LNA nucleotides and oligonucleotides are generally described in WO 99/14226, WO 00/56746, WO 00/56748, WO 00/66604, WO 00/125248, WO 02/28875, WO 2002/094250 and WO 2003/085110, all incorporated herein by reference.

In one embodiment, the oligomeric compound comprises one or more UNAs (unlocked nucleic acid) monomers, which are acyclic derivatives of RNA lacking the C2′-C3′-bond of the ribose ring of RNA. Synthesis of phosphoramidite UNA building blocks of the nucleobases adenine, cytosine, guanine, and uracil and their incorporation into RNA strands are described in, e.g., Langkjaer et al., Bioorg Med Chem. 2009 Aug. 1; 17(15):5420-5, herein incorporated by reference in its entirety. Other modified nucleotides suitable for use in the oligomeric compounds include, without limitation, 2′-fluororibose.

The oligomeric compound may also comprise internucleoside linkage modifications, e.g., selected from phosphorothioate, 3′-methylenephosphonate (i.e. 3′-O-methylphosphonate internucleotide linkage), 5′-methylenephosphonate (i.e. 5′-O-methylphosphonate internucleotide linkage), 3′-phosphoroamidate (i.e. N-3′-phosphoroamidate internucleotide linkeage) and 2′-5′-phosphodiester (i.e. 2′-5′-phosphodiester internucleotide linkage). Especially preferred are phosphorothioate linkages. Accordingly, in some embodiments, the oligomeric compound comprises at least two nucleotides and/or nucleotide analogues linked by a phosphorothioate group. Preferred are, for example, oligomeric compounds wherein the 2, 3, 4, 5, or 6 terminal nucleotides are linked with phosphorothioate linkages, preferably wherein the terminal 5 nucleotides at the 5′ and 3′ terminus are linked with phosphorothioate linkages.

The oligomeric compound may also comprise or consist of an phosphorodiamidate Morpholino oligomer (PMO), also known as “Morpholino” or “Morpholino oligomer”, where the DNA bases are attached to a backbone of methylenemorpholine rings linked through phosphoro-diamidate groups. Their design, preparation and properties have been described by, e.g., Summerton et al., Antisense & Nucleic Acid Drug Development 1997:7 (3): 187-95, herein incorporated by reference.

Particularly contemplated are oligomeric molecules where the target binding domain, the oligomeric compound, or both, is an RNA molecule selected from an small interfering RNA (siRNA), short hairpin RNA (shRNA), a guide RNA (gRNA), single guide RNA (sgRNA), or CRISPR RNA (crRNA) molecule.

RNA Interference (RNAi)

RNA molecules may inhibit gene expression or translation by neutralizing the targeted mRNA molecules. Without being bound by theory, it is believed that long double stranded RNA introduced into cells is broken down into double stranded short interfering RNAs (siRNAs) comprising a sense strand and an antisense strand by a Type III endonuclease known as Dicer. Dicer, a ribonuclease-III-like enzyme, processes these dsRNA into 19-23 base pair short interfering RNAs with characteristic two base 3′ overhangs. These siRNAs are then incorporated into an RNA-induced silencing complex (RISC) where one or more helicases unwind the siRNA duplex, enabling the complementary antisense strand to guide target recognition. Upon binding to the appropriate target mRNA, one or more endonucleases within the RISC cleave the target to induce silencing (Elbashir, et al., (2001) Genes Dev. 15:188).

Accordingly, RNAi-based oligomeric compounds of the invention include single-stranded RNA comprising a target-binding region that interacts with a target RNA sequence, e.g., in nNav1.5 mRNA, to direct the cleavage of the target RNA, as well as double-stranded versions thereof (dsRNA).

Preferably, the oligomeric compound is an siRNA molecule, optionally in double-stranded form, i.e., a duplex. The siRNA target binding sequence may comprise or consist of 15, 16, 17, 18, 19, 20, 21, 22, 23, 24, 25, 26, 27, 28, 29 or 30 or more contiguous nucleobases. Accordingly, siRNA target binding sequences of 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length are contemplated. In one particular embodiment, the antisense siRNA strand comprises or consists of 18-20 nucleotides, such as 19 nucleotides.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30 or more base pairs in length.

In addition to the duplex structure, an RNAi may comprise one or more nucleotide overhangs. For example, in one embodiment, the antisense strand, sense strand, or both of a duplex has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide, such as at least 2, at least 3, at least 4, at least 5, at least 6, at least 7, at least 8, at least 9, at least 10, at least 20, at least 23 or more unpaired nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.”

Preferred oligomeric compounds comprise or consist of an RNA molecule comprising a target binding region complementary, preferably directly complementary, to GAGUCCUGAGAGCUCUAAA (NESO; SEQ ID NO:15); CUAGGCAAUUUGUCGGCUC (Neo1; SEQ ID NO:13), or UAUCAUGGCGUAUGUAUCA (Neo2; SEQ ID NO:14), optionally in double-stranded form.

Other preferred oligomeric compounds comprise or consist of an RNA molecule comprising a target binding sequence (in 5′→3′ direction, where the 5′-end is phosphorylated and the 3′-end is hydroxylated) selected from GAGUCCUGAGAGCUCUAAA (NESO; SEQ ID NO:15); CUAGGCAAUUUGUCGGCUC (Neo1; SEQ ID NO:13), and UAUCAUGGCGUAUGUAUCA (Neo2; SEQ ID NO:14), optionally in double-stranded (duplex) form.

Particularly preferred oligomeric compounds comprise or consist of an RNA molecule (in 5′→3′ direction, where the 5′-end is phosphorylated and the 3′-end is hydroxylated) selected from GAGUCCUGAGAGCUCUAAA (NESO; SEQ ID NO:15); CUAGGCAAUUUGUCGGCUC (Neo1; SEQ ID NO:13), and UAUCAUGGCGUAUGUAUCA (Neo2; SEQ ID NO:14) in double-stranded (duplex) form.

Oligomeric compounds that are also contemplated include double-stranded siRNA molecules comprising a sense strand and an antisense strand, wherein the antisense strand comprises a target-binding domain directly complementary to a contiguous portion of 15, 16, 17, 18, 19, 20, 21, 22, 23, 24, 25, 26, 27, 28, 29 or 30 or more nucleobases, such as 19 nucleobases, of SEQ ID NO:3, particularly a contiguous portion of 15, 16, 17, 18, 19, 20, 21, 22, 23, 24, 25, 26, 27, 28, 29 or 30 or more nucleobases of the portion corresponding to residues 797 to 896 of SEQ ID NO:3.

The target-binding domain may, for example, comprise 19 residues directly complementary to a contiguous portion starting at nucleotide 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 862, 864, 865, 866, 867, 868, 869, 870, 871, 872, 873, 874, 875, 876, 877 or 878 of SEQ ID NO:3.

In other embodiments, the target-binding domain may comprise 19 RNA residues directly complementary to a DNA or RNA sequence directly complementary to a contiguous portion starting at nucleotide 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 862, 864, 865, 866, 867, 868, 869, 870, 871, 872, 873, 874, 875, 876, 877 or 888 of SEQ ID NO:3.

Functional Characteristics

The oligomeric compounds of the invention may also or alternatively be characterised by their ability to reduce the Nav1.5 mRNA level, Nav1.5 expression level, Nav1.5 activity and/or cancer cell invasiveness.

Suitable assays can be found in the present Example.

For example, cells of a cancer cell line, e.g., SW620 cells, can be transfected with oligomeric compound using a cationic liposome formulation such as, e.g., Lipofectamine 2000 to effect transfection according to the manufacturer's instructions. For example, a final concentration of 40 nM oligomeric compound (e.g., in the form of siRNA targeting Nav1.5) can be achieved. A suitable control may be, for example, an oligomeric compound targeting an irrelevant or non-existent target sequence or simply the absence of any oligomeric compound. After a predetermined period after transfection, such as about 24 h, about 48 h, about 72 h, about 96 h, or about 120 h, such as about 90 h, the relevant assay can be performed.

The mRNA level can be determined using conventional PCR technology and the disclosed primer sets for nNav1.5 (SEQ ID NOS:4 and 5) or general Nav1.5 (SEQ ID NOS:6 and 7), or the expression level of nNav1.5 and/or aNav1.5 on the cell surface determined using suitable antibodies specific for the desired protein. To determine the expression level of nNav1.5 in cancer cells and/or on the surface of cancer cells, NESO-pAb antibody can advantageously be used. Typically, an oligomeric compound according the invention can reduce the mRNA level, the protein expression level, or both, of the target Nav1.5 protein by at least about 10%, 20% or more, such as by at least 30%, 40%, 50%, 60%, 70% or 80% as compared to a control.

The function of Nav1.5 can be studied using the electrophysiological assay described in Example 1, with details as described previously (e.g. Laniado et al., 1997; 2001; Fraser et al., 2003a; Grimes et al, 1995). For example, patch pipettes (tip resistances, ˜5 MΩ) can be filled with a solution designed to block the outward K⁺ currents; e.g., (in mM): NaCl 5, CsCl 145, MgCl₂ 2, CaCl₂ 1, HEPES 10 and EGTA 11, adjusted to pH 7.4 with 1 M CsOH. The intracellular free Ca²⁺ concentration can be estimated, e.g., ˜15 nM (Laniado et al., 2001). Whole-cell membrane currents can be recorded from cells that appear ‘isolated’ in culture, e.g., using an Axopatch 200B amplifier (Axon Instruments, Calif., USA). Analogue signals can be filtered at 10 kHz using a low-pass Bessel filter, and series resistance errors can be compensated by >90%. Electrophysiological signals may be sampled at 50 kHz and digitised, e.g., using an interface such as Digidata 1200. Data acquisition and analysis of whole-cell currents can then be performed, e.g., using suitable software such as pClamp software (Axon Instruments). A holding potential of −100 mV may be applied. Standard voltage-clamp protocols were used to study the electrophysiological properties of the VGSC currents. All routine recordings can be done after a suitable time of incubation, e.g., at 24 hours after (re)plating and 24 hours of serum starvation (to match the condition of the invasion assays). Conductance-voltage relationships and other relevant parameters for evaluating VGSC currents can then be calculated as described in Example 1, using equations (I) to (III), with further details provided in Onkal et al. (2008). Other suitable assays for evaluating the effect of the oligomeric compound on the VGSC current are known in the art (see, e.g., Rajamani et al., 2016). Typically, an oligomeric compound of the present invention results in a significant reduction in peak VGSC current density relative to control and/or significantly reduces the proportion of cells demonstrating VGSC currents, e.g., by at least about 10%, 20% or more, such as by at least 30%, 40%, 50%, 60%, 70% or more as compared to a control.

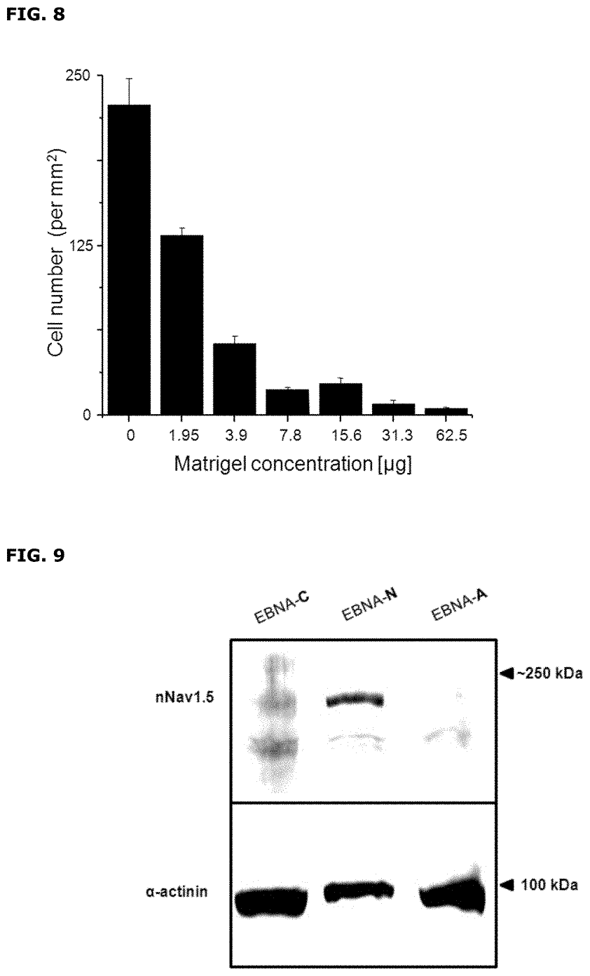

The ability of an oligomeric compound to reduce the invasiveness of cancer cells can be determined using the assay described by (Fraser et al., 2005), following optimization of the cell number versus the Matrigel concentration (see FIG. 8). Accordingly, (i) insert filters (with 8 μm pores) can be coated with 50 μl of 0.21 mg/ml Matrigel (BD Biosciences, Bedford, Mass., USA); (ii) a chemotactic gradient can be 0.1-10% FBS; and (iii) the cells can be serum-starved for 24 hours and (iv) about 10⁵ cells can be seeded onto each filter. After a suitable period of incubation, such as 48 hours, the insert can be swabbed and then stained with crystal violet. The invaded cells in 12 non-overlapping fields of view can then be counted, e.g., under ×400 magnification. “Invasiveness” can then be calculated as the number of invaded cells normalized to the largest value observed amongst the different treatment conditions in given experimental sets. Typically, an oligomeric compound of the present invention results in a significant reduction of invasiveness, e.g., by at least about 10%, 20% or more, such as by at least 30%, 40%, 50%, 60%, 70% or 80% as compared to a control. This may be observed both under normoxic and hypoxic conditions.

Additional or alternative cellular assays that may also be used for evaluating the effect of oligomeric compounds on Nav1.5-expressing cancer cells can be found in WO 2018/146313 (Celex GmbH) and W02012/049440 (Celex Oncology Ltd.), e.g., assays for studying cell motility, cell adhesiveness, invasiveness, etc.

Accordingly, in separate and specific embodiments, an oligomeric compound of the invention may reduce or prevent metastatic behaviour of cancer cells, such as colorectal cancer cells, e.g., colon cancer cells, by, for example:
(a) reducing the invasiveness of cancer cells, optionally under both normoxic and hypoxic conditions;
(b) reducing the motility of cancer cells, optionally under hypoxic but not normoxic conditions;
(c) decreasing cancer cell expression of at least one VGSC, optionally under both normoxic and hypoxic conditions;
(d) reducing the ability of cancer cells to migrate; or
(e) a combination of two or more of (a) to (d), e.g., (a) and (b), (a) and (c), (a) and (d), (b) and (c), (b) and (d), (c) and (d), (a) to (c) or (a) to (d).

Delivery Vehicles and Compositions

The delivery of an oligomeric compound of the invention to a cancer cell or tumor, e.g., a cancer cell or tumor in a subject to be treated according to the invention, can be performed in a number of different ways.

The oligomeric compound can be formulated in a composition, typically a pharmaceutical composition, for administration by any suitable route to the patient, including, but not limited to, oral, buccal, sublabial, sublingual, rectal, intravenous, subcutaneous, intradermal, intramuscular, transdermal and intranasal administration and/or direct administration to a tumour, such as a primary tumour. Local administration may be particularly useful, e.g., by directly injecting a pharmaceutical composition comprising the oligomeric compound into or near a tumor or suspected tumor site. Sustained-release systems may also be used, particularly so as to release the compound over a prolonged period of time. Delivery can also be performed by systemic administration of a composition, such as a pharmaceutical composition, comprising the oligomeric compound to a subject, e.g., a cancer patient. Delivery can also or alternatively be performed indirectly, by administering one or more vectors that encode an oligomeric compound, particularly an oligonucleotide.

Accordingly, in some embodiments, the oligomeric compound is expressed from transcription units inserted into a DNA or RNA vector, optionally wherein the vector further comprises one or more expression control sequences. Expression can be transient (on the order of hours to weeks) or sustained (weeks to months or longer), depending upon the specific construct used and the target tissue or cell type. The transcription unit can be introduced as a linear construct, a circular plasmid, or a viral vector, which can be an integrating or non-integrating vector.

Suitable RNAi expression vectors are generally DNA plasmids or viral vectors. An RNAi-based oligomeric compound can be transcribed from a promoter on an expression vector. In the case of a double-stranded molecule, the two separate strands can be expressed from the same or two different expression vectors co-introduced (e.g., by transfection or infection) into a target cell. Alternatively, a dsRNA molecule can be expressed as an inverted repeat polynucleotide joined by a linker polynucleotide sequence such that the dsRNA has a stem and loop structure.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Alternatively, the construct can be incorporated into vectors capable of episomal replication, e.g. EPV and EBV vectors. Constructs for the recombinant expression of an RNAi will generally require regulatory elements, e.g., promoters, enhancers, etc., to ensure the expression of the RNAi in target cells.

In one particular embodiment, the plasmid or viral vector encoding an oligomeric compound which is a gRNA further encodes a transactivating crRNA (tracrRNA) and a CRISPR-associated enzyme selected from Cas9 and Cpf1, or both. This type of vector may, for example, be used to disrupt the neonatal version of exon 6 in the Nav1.5 gene using the gRNA as a target binding region to target the CRISPR system to genomic nNav1.5 DNA.

Pharmaceutical compositions comprising the oligomeric compounds of the present invention and/or their delivery vehicles or vectors include, but are not limited to, solutions, emulsions, liposome-containing, and lipid-nanoparticle (LNP)-containing formulations. These compositions may be generated from a variety of components that include, but are not limited to, preformed liquids, self-emulsifying solids and self-emulsifying semisolids.

Positively charged cationic delivery systems facilitate binding of (typically) negatively charged oligonucleotide-based oligomeric compounds or plasmid vectors, and enhance interactions at the negatively charged cell membrane to permit efficient uptake of the oligomeric compound or vector by the cell.

Cationic lipids, dendrimers, or polymers can either be bound to an oligomeric compound, or induced to form a vesicle or micelle that encases the oligomeric compound. The formation of vesicles or micelles further prevents degradation of the compound when administered systemically.

In one embodiment, the oligomeric compound is encapsulated within liposomes or complexed to liposomes, in particular to cationic liposomes. Liposomes traditionally include one or more rings of lipid bilayer surrounding an aqueous pocket. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm. Liposomes which are pH-sensitive or negatively charged, entrap nucleic acids rather than complex with it. Suitable lipids for liposomes include, without limitation, neutral lipids (e.g., dioleoylphosphatidyl DOPE ethanolamine, dimyristoylphosphatidyl choline DM PC, distearolyphosphatidyl choline), negative (e.g., dimyristoylphosphatidyl glycerol DM PG) and cationic lipids, such as those exemplified below.

The oligomeric compounds can also be provided as micellar formulations, with “micelles” referring to a molecular assembly in which amphipathic molecules are arranged in a spherical structure such that all the hydrophobic portions of the molecules are directed inward, leaving the hydrophilic portions in contact with the surrounding aqueous phase. Exemplary micelle forming compounds include lecithin, hyaluronic acid, pharmaceutically acceptable salts of hyaluronic acid, glycolic acid, lactic acid, and so forth.

In a particular embodiment, an oligomeric compound of the invention is comprised in an LNP, which may assume a micelle-like structure, encapsulating drug molecules in a non-aqueous core. LNPs typically contain a cationic lipid, a non-cationic lipid, and a lipid that prevents aggregation of the particle (e.g., a PEG-lipid conjugate). LNPs are very useful for systemic applications, as they protect the oligonucleotide from nuclease-mediated degradation and exhibit extended circulation lifetimes following intravenous injection, accumulating at distal sites, which may include a tumor site.

Suitable cationic lipids include, but are not limited to, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), and N,N-dimethyl-2,3-dioleyloxy)propylamine (DODMA). The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), and palmitoyloleoylphosphatidylcholine (POPC). The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. In some embodiments, the nucleic acid-containing LNP further includes cholesterol.

Nucleic acid-containing lipid particles (LNPs) and their methods of preparation are disclosed in, e.g., U.S. Pat. Nos. 5,976,567; 5,981,501; 6,534,484; 6,586,410; 6,815,432; U.S. Publication No. 2010/0324120 and PCT Publication Nos. WO 96/40964 and WO 00/03683, each of which is hereby incorporated by reference. These and other useful formulation or drug delivery techniques suitable for oligomeric compounds, including RNAi and siRNA, are disclosed in WO 17/023660, which is also incorporated herein by reference.

The compositions, such as pharmaceutical compositions, of the present invention may be formulated into any of many possible dosage forms such as, but not limited to, tablets, capsules, gel capsules, liquid syrups, soft gels and suppositories. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. A suspension may also contain thickeners and/or stabilizers.

Therapeutic Applications

An oligomeric compound of any aspect or embodiment as described herein may be used as a medicament, e.g., for treatment of cancer. Suitable patients include mammalian patients, such as humans, monkeys, rabbits, dogs, cats, cows, horses, pigs, mice and rats, suffering from cancer. Preferably, the patient is a human patient, such as an adult human patient.

In one aspect, the invention provides a method of treating a cancer comprising cancer cells that express the neonatal form of human Nav1.5 (nNav1.5), comprising administering to a subject suffering from said cancer an oligomeric compound comprising a target binding domain that is specifically hybridisable to mRNA or genomic DNA encoding Nav1.5, wherein the oligomeric compound reduces the level of nNav1.5 mRNA in the cancer cells, the level of nNav1.5 expressed on the surface of the cancer cells, or both.

In one aspect, the invention provides a method of treating a cancer selected from colorectal cancer, breast cancer, lung cancer, ovarian cancer or neuroblastoma, or a combination of any thereof, wherein the oligomeric compound comprises a target binding domain that is specifically hybridisable to messenger RNA (mRNA) or genomic DNA encoding nNav1.5. in some embodiments, the colorectal cancer is colon cancer.

As used herein, “treating” or “treatment” of a cancer includes, but is not limited to, reducing metastatic behaviour of a cancer, preventing metastatic behaviour of a cancer, reducing pain sensation, reducing the invasiveness of a cancer, reducing the overall aggressiveness of the cancer, or any combination thereof. So, in separate and specific embodiments, a method of treatment according to the invention may (i) reduce metastatic behaviour of the cancer, (ii) prevent metastatic behaviour of the cancer, (iii) reduce pain sensation in a patient suffering from the cancer, (iv) reduce the invasiveness of the cancer, or (v), and combination of two or more of (i) to (iv).

Progression of metastatic cancer, such as breast, colon and prostate cancer, is generally considered as comprising at least some of five main phases, as follows:
1. Genesis, namely the initial transformation of a normal cell into a cancer cell;
2. Proliferation, namely increase in the number of cancer cells to form a primary tumour of increasing size, typically with a smooth and well defined surface;
3. Switching, during the genesis or proliferation phase, from a condition in which the cancer cells have no potential for invasive or metastatic behaviour to a condition in which they do, typically characterised by a dissolving and diffuse boundary of the cancer;
4. Detachment of cancer cells from the primary tumour followed by movement of those detached cells into surrounding regions of tissue within the same organ towards the circulation system;
5. Metastasis, namely the movement of the detached cells through the circulation (blood or lymph) to other organs to create secondary tumours in those other organs.

It should be noted, however, that metastasis may occur without an initial proliferative phase. In such cases, metastases may be found in a patient without an identifiable primary tumour.

By “reducing metastatic behaviour” of cancer, it is intended a reduction of any behaviour associated with the movement of detached cancer cells through the circulation (blood or lymph) to accumulate and/or create secondary tumours in other organs or locally invade surrounding tissues. Typically, the patient is in phase 3, 4 or 5, such as in phase 4 or 5. Reducing metastatic behaviour may, for example, include one or more of (i) reducing transcription, translation and/or expression of neonatal and/or adult Nav1.5 in cancer cells as compared to a control, typically reducing at neonatal Nav1.5 (nNav1.5); (ii) reducing cancer cell invasiveness; (iii) reduce peak VGSC current density in cancer cells; (iv) reduce the proportion of cancer cells demonstrating VGSC currents; (v) reducing cancer cell motility (e.g., reduced lateral motility), (vi) reducing cancer cell migration (e.g., transverse migration), and (vii) reducing the persistent part of the VGSC current without eliminating the transient part. The VGSC may, for example, be Nav1.5 (in adult and/or neonatal form), such as nNav1.5. “Motility” reflects the ability of the tumour cells to initially move to and through the basement membrane into the surrounding tissue; “invasiveness” of the cells reflects the ability of tumour cells which have entered the surrounding tissue to move through that tissue towards the circulation system; and “migration” reflects the ability of the tumour cells to migrate from that tissue into the circulatory system via the walls thereof.

By “preventing metastatic behaviour” of cancer, it is intended to refer prophylactic treatment of a cancer patient at risk for, but not yet diagnosed with, a metastatic disease, so as to prevent or reduce the risk for a metastatic behaviour of the cancer as described above. Typically, the patient is in phase 1, 2 or 3. Preventing metastatic behaviour may, for example, include preventing or reducing the expression of one or more of Nav1.5 in adult and/or neonatal form, such as nNav1.5.

The term “benign state” as used herein refers to a tumour or cancer in phase 1 or 2. As used herein, tumours may also or alternatively be characterized as being in a benign state if they (a) do not invade nearby tissue (invasiveness); (b) do not metastasize (spread) to other parts of the body; (c) tend to have clear boundaries; and/or (d) grow slowly.

The term “malignant state” herein refers to a tumour or cancer in phase 3, 4 or 5.

By “reducing the overall aggressiveness of a cancer”, it is intended a reduction of any behaviour associated with the progression of cancer, in quantitative or qualitative terms. In some embodiments, reducing the aggressiveness of a cancer refers to the reversal of a cancer in any one of phase 3, 4 or 5 to a lower-number phase, including, but not limited to, from phase 3 to phase 2 or lower, from phase 4 to phase 3 or lower, and from phase 5 to phase 4 or lower. In some embodiments, reducing the aggressiveness of a cancer refers to the reversal of a cancer or tumour in a malignant state to a cancer or tumour in a benign state. In some embodiments, by “reducing the overall aggressiveness of a cancer”, it is intended a reduction of a cancer to non-metastatic but not necessarily non-invasive state.

By “reducing the invasiveness of a cancer”, it is intended a significant reduction of the invasiveness of the cancer cells under predetermined conditions, e.g., normoxic or hypoxic conditions. Examples of assays suitable to determine invasiveness are provided elsewhere herein (see, e.g., the section entitled “Functional characteristics”). A significant reduction of invasiveness includes, e.g., a reduction by at least about 10%, 20% or more, such as by at least 30%, 40%, 50%, 60%, 70% or 80% as compared to a control.

In some embodiments of the methods of the invention, the oligomeric compound is administered in a therapeutically effective amount or dose. By “therapeutically effective amount”, “therapeutically effective dose”, it is intended an amount or dosage of compound of oligomeric compound that, when administered to a patient suffering from cancer brings about a positive therapeutic response with respect to treatment of the patient, such as, e.g., reduction of metastatic behaviour of the cancer, prevention of metastatic behaviour of the cancer, reduction of pain, or the like.

The compound is administered to the patient in a therapeutically effective amount for the intended purpose, and with a frequency and for a period of time determined by a trained physician. Estimates of effective dosages and in vivo half-lives for the individual oligomeric compounds encompassed by the invention can be made using conventional methodologies or on the basis of in vivo testing using an appropriate animal model, as described elsewhere herein. The pharmaceutical compositions of the present invention can be administered in a number of ways (also described elsewhere herein) depending upon whether local or systemic treatment is desired and upon the tumor or cancer to be treated.

For example, in some embodiments, the oligomeric compound of the invention, such as a double-stranded siRNA molecule according to any embodiment described herein, is administered to a patient in a dosage of at least about 2, such as at least about 3, such as at least about 4, such as at least about 5 mg per kg body weight of the patient. In general, a suitable dosage will be in the range of about 0.0001 to about 200 mg oligomeric compound per kg bodyweight, such as from about 0.1 to about 10 mg or from about 1 to about 50 mg per kg bodyweight per single dose and/or per day. Suitable treatment regimens may comprise repeating the administration at least once, such as at daily, weekly or monthly intervals, typically until a therapeutic benefit or response is observed. Delivery vehicles for the oligomeric compound, including sustained-release formulations, may be employed as needed, as described elsewhere herein.

Typically, a cancer selected for treatment according to the invention comprises Nav1.5-expressing cells or is a cancer associated with a known risk for Nav1.5 expression and thereby metastatic behaviour. Preferably, a cancer selected for treatment according to the invention comprises cancer cells expressing nNav1.5 or is a cancer associated with a known risk for nNav1.5 expression and thereby metastatic behaviour. Table 1 shows links that have been found between some particular cancer forms and their VGSC expression, particular with respect to Nav1.5. These include, but are not limited to, colorectal cancer, breast cancer, lung cancer, ovarian cancer, astrocytoma or neuroblastoma, and combinations of any thereof. As used herein, colorectal cancer can in some embodiments be characterized as colon cancer or rectal cancer, typically depending on the tissue origin of the cancer. Unless contradicted by context, however, colorectal cancer and colon cancer may be used interchangeably. In preferred embodiments, the cancer is colorectal cancer, colon cancer, or both. In some embodiments, one or more tumours in the patient are hypoxic or are at risk for being hypoxic. In some embodiments, one or more tumours in the patient are expected or determined by the trained physician to be hypoxic. The presence of hypoxia can be determined by a variety of techniques known in the art, including, but not limited to, magnet resonance (MR) imaging (see, e.g., Abadjian et al., Adv Exp Med Biol. 2017;1036:229-257) or staining a sample of tumour tissue with pimonidazole (see, e.g., Wilson and Hay, Nature Reviews Cancer 2011; 11: 393-410).

In some embodiments, the patient is suffering from a cancer comprising Nav1.5-expressing cancer cells. Such a cancer may, for example, be identified by immunohistochemical or analysis of a cancer cell-containing sample (such as a tumour biopsy or blood sample) obtained from the patient, using detectable monoclonal or polyclonal antibodies specific for nNav1.5, aNav1.5 or both to detect the expression of Nav1.5 by the cancer cells. In one embodiment, the cancer cells express both aNav1.5 and nNav1.5. In a specific embodiment, the cancer expresses Nav1.5 in adult and/or neonatal form, such as, e.g., neonatal Nav1.5. Preferably, the cancer cells express, at least predominantly, Nav1.5 in neonatal form.

In some embodiments, the treatment methods described herein comprises a step of determining that the cancer comprises cells expressing Nav1.5, e.g., neonatal and/or adult Nav1.5, preferably neonatal Nav1.5, typically conducted prior to administering the oligomeric compound. This can be performed by taking a sample from a tumour in the subject contemplated for treatment, e.g., a tumour biopsy, and analysing the tumour sample or tumour cells for the relevant Nav1.5 mRNA, expression of Nav1.5 protein, or both. Suitable assays for doing this are described elsewhere herein. In a preferred embodiment, the treatment method comprises a step of determining that the cancer comprises cells expressing nNav1.5 prior to administering the oligomeric compound.

A VGSC (nNav1.5) expressing cancer is in phase 3, 4 or 5 as described above.

In one embodiment, the patient is in stage 3, 4 or 5, such as in phase 4 or 5. In one embodiment, the cancer is in stage 1, 2, or 3, such as in phase 1 or 2.

In one embodiment, the cancer is in phase 3. A patient suffering from a cancer in phase 3 has typically not been diagnosed with metastatic disease, but is at risk for metastatic behaviour of the cancer, i.e., progression to phase 4 or 5. A patient suffering from a cancer in phase 3 may thus be treated according to the invention to prevent metastatic behaviour of the cancer.

In one embodiment, the cancer is in phase 4. A patient suffering from a cancer in phase 4 may not have been diagnosed with metastatic disease, but the cancer has progressed towards metastatic behaviour. A patient suffering from a cancer in phase 4 may thus be treated according to the invention to reduce metastatic behaviour of the cancer.

In one embodiment, the cancer is in phase 5. A patient suffering from a cancer in phase 5 may have been diagnosed with metastatic disease, and the cancer is characterized by metastatic behaviour. A patient suffering from a cancer in phase 5 may thus be treated according to the invention to reduce metastatic behaviour of the cancer.

In some embodiments, the patient may be suffering from a cancer associated with a risk for VGSC-expression and/or metastatic behaviour, but VGSC-expression (in particular nNav1.5 expression) and/or metastatic behaviour has not yet been determined. Cancers that are prone to metastatic behaviour include, for example, colon cancer, breast cancer, lung cancer, and ovarian cancer. For example, an immunohistochemical analysis of a cancer cell-containing sample such as a tumour biopsy or blood sample obtained from the patient may have indicated that the tumour cells in the sample did not express the nNav1.5 or other VGSCs tested for. The cancer may thus be in phase 1 or (more likely) in phase 2.

In one embodiment, the cancer is in phase 2. A patient suffering from a cancer in phase 2 has typically not been diagnosed with metastatic disease, but is at risk for VGSC (nNav1.5) expression and metastatic behaviour of the cancer, i.e., progression to phase 3, 4 or higher.

A patient suffering from a cancer in phase 2 may thus be treated according to the invention to prevent nNav1.5-expression or metastatic behaviour of the cancer.

A patient suffering from a cancer in any one of phase 1-5, such as in any one of 2-5, may also suffer from pain caused by the cancer, e.g., by a primary tumour, and may thus be treated according to the invention to reduce pain sensation.

In one embodiment, when used in a method according to the invention, the compound reduces or prevents metastatic behaviour in nNav1.5-expressing cancer without killing the cancer cells.

In one embodiment, when used in a method according to the invention, the compound reduces or prevents metastatic behaviour in nNav1.5-expressing cancer without substantially affecting proliferation of the cancer cells.

In one embodiment, treatment of cancer cells with the compound results in cancer cell expression of Nav1.5 in neonatal form being significantly lower than that of a control, such as a predetermined control value, cancer cells not exposed to the compound or cancer cells exposed to a reference compound. In one embodiment, treatment of cancer cells with the compound results in the invasiveness, motility and/or ability to migrate of cancer cells treated with the compound being significantly lower than that of a control, such as a predetermined control value, cancer cells not exposed to the compound or cancer cells exposed to a selected reference compound.

In a particular aspect, the method of treatment further comprises a second therapeutic agent to the subject. In a particular embodiment, the second therapeutic agent is not a VGSC blocker.

The invention is further illustrated by the following Example, which should not be interpreted as limiting. It is understood that modifications can be made in the procedures set forth without departing from the spirit of the invention.

### EXAMPLE 1

The main aims of the present Example were (1) to quantify nNav1.5 mRNA and protein expression in several CRCa cell lines; to compare the relative contributions of nNav1.5 vs. aNav1.5 (2) to the VGSC current and (3) to the VGSC-dependent control of invasiveness. In addition, (4) we determined the impact of hypoxia on invasiveness and its dependence on nNav1.5. Finally (5), we evaluated the possible anti-invasive effects of ranolazine, a blocker of hypoxia-associated VGSC activity.

Materials and Methods

Cell Lines and Basal Culture Conditions

Three different human CRCa cell lines were used: HT29, HCT116 and SW620 (Brattain et al., 1981; Fogh, 1975; Leibovitz et al., 1976). Most experiments were done on the SW620 cell line derived originally from a lymph-node metastasis and later shown to have ‘sternness’ (Kawamoto et al., 2010; Leibovitz et al., 1976). All cells were cultured in Roswell Park Memorial Institute formulation 1640 (RPMI 1640) medium (Invitrogen, Paisley, UK) supplemented with 4 mM L-glutamine and 10% foetal bovine serum (FBS) (Invitrogen). Culturing was in a humidified incubator at 37° C. with 100% relative humidity and 5% CO₂ (Fraser et al., 2005). For hypoxia, cells were maintained as above but in 1% O₂ for up to 120 h in a dedicated incubator (Micro Galaxy, RS Biotech Laboratory Equipment Ltd, Irvine, UK).

Electrophysiology and Curve Fitting of Data

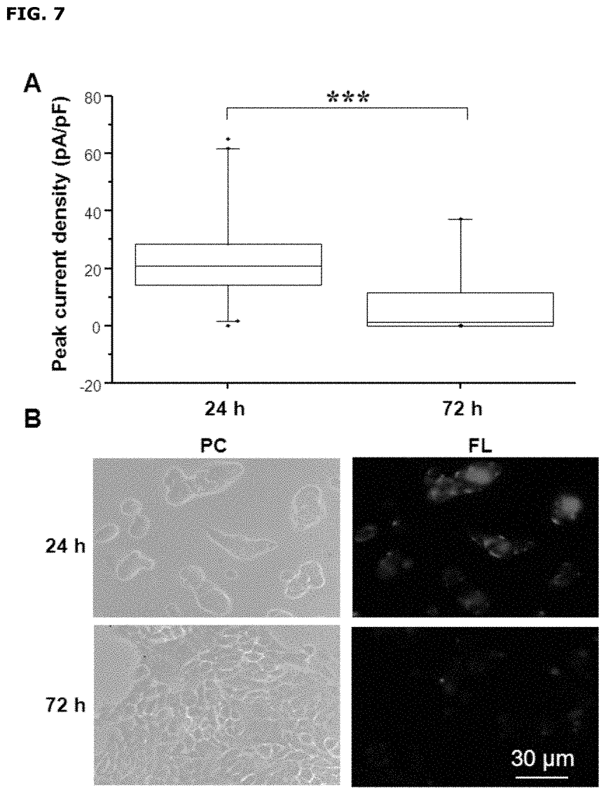

Details of the patch pipettes, solutions, and the whole-cell recording protocols were as described previously (e.g. Laniado et al., 1997; 2001; Fraser et al., 2003a; Grimes et al, 1995). In brief, patch pipettes (tip resistances, ˜5 MΩ) were filled with a solution designed to block the outward K⁺ currents; the composition was as follows (in mM): NaCl 5, CsCl 145, MgCl₂ 2, CaCl₂ 1, HEPES 10 and EGTA 11, adjusted to pH 7.4 with 1 M CsOH. The estimated intracellular free Ca²⁺ concentration was ˜15 nM (Laniado et al., 2001). Whole-cell membrane currents were recorded from cells that appeared ‘isolated’ in culture, using an Axopatch 200B amplifier (Axon Instruments, Calif., USA). Analogue signals were filtered at 10 kHz using a low-pass Bessel filter, and series resistance errors were compensated by >90%. Electrophysiological signals were sampled at 50 kHz and digitised using an interface (Digidata 1200). Data acquisition and analysis of whole-cell currents were performed using pClamp software (Axon Instruments). A holding potential of −100 mV was applied, unless indicated otherwise. Standard voltage-clamp protocols were used to study the electrophysiological properties of the VGSC currents. There was a noticeable decrease in current amplitude with time in culture, also apparent in immunocytochemistry (FIG. 7). All routine recordings were done 24 hours after (re)plating and 24 hours of serum starvation (to match the condition of the invasion assays).

Conductance-voltage relationships were determined using the equation:

G=I/(V−Vᵣₑᵥ) (I),

where G is the conductance; I the current amplitude; V the test pulse; and Vᵣₑᵥ the theoretical Na⁺ reversal potential. Normalized curves for voltage dependence of steady-state activation and inactivation were fitted to a Boltzmann function of the form:

G=Gₘₐₓ/[1−exp(Vₘ−V_{1/2})/k] (II)

where Gₘₐₓ is the maximal conductance; Vₘ is the membrane voltage; V_{1/2} is the voltage at which the current is half activated/inactivated, and k is the slope factor of voltage sensitivity. For the time course of recovery from inactivation, the data was plotted as a function of recovery time and fitted to the following single exponential equation:

I=A exp(−t/t)+C (III)

where I is normalized current, t is time, t is the time constant, A is the amplitude of the normalized current, and C is the asymptote. More details can be found in Onkal et al. (2008).

Pharmacology

All pharmacological agents were obtained from Sigma-Aldrich (Poole, UK), except where specified. Tetrodotoxin (TTX) (Alomone Labs, Jerusalem, Israel) was prepared as a stock solution of 3132 μM in normal culture medium and used at a final working concentration of 20 μM. Since Nav1.5 is TTX-resistant and neonatal and adult isoforms do not differ in their TTX sensitivity, this concentration of TTX would block >>80% of currents generated by either Nav1.5 splice variant (Onkal et al., 2008). Ranolazine was prepared as a stock solution of 2 mM in normal culture medium and used in the concentration range 1-10 μM. Aconitine, used in preliminary experiments, was prepared in dimethyl sulfoxide (DMSO) at a stock concentration of 100 mM. The final working concentration of 100 μM contained 0.1% DMSO. The control solution was 0.1% DMSO.

Polymerase Chain Reactions

Steps for polymerase chain reactions (PCRs) were as described before (Fraser et al., 2005). Conventional PCRs were performed using HotStarTaq Plus technology (Qiagen Ltd, Manchester, UK) in a Primus PCR machine (MWG-Biotech, Eurofins Genomics, Ebersberg, Germany). Quantitative real-time PCRs were carried out utilising SYBR Green technology (Qiagen) and a DNA Engine Opticon 2 system (MJ Research, Bio-Rad Laboratories Ltd, Hamel Hempstead, UK). Duplicate reactions on each sample were carried out simultaneously for target and reference genes. Control PCRs were carried out routinely by including non-target (−RT) reactions and monitoring melting curves. The primer pairs used were as follows:

1) Neonatal Nav1.5 (SCN5A):
(F; SEQ ID NO: 4)
5′-CTGCACGCGTTCACTTTCCT-3′;
(R; SEQ ID NO: 5)
5′-GACAAATTGCCTAGTTTTATATTT-3′;
(J. K. J. Diss, unpublished).
2) General Nav.15 (SCN5A):
(F: SEQ ID NO: 6)
5′-CTGCACGCGTTCACTTTCCT-3′;
(R; SEQ ID NO: 7)
5′-CAGCCAGCTTCTTCACAGACT-3′;
(J. K. J. Diss, unpublished). These targeted the
spliced region (DI:S3-S4) encapsulatingboth
nNav1.5 and aNav1.5. The resultingPCR products
were sequenced (MWG-Biotech). More details of the
primers can be found in Guzel (2012).
3) Control gene-1 (CYB5R3):
(F; SEQ ID NO: 8)
5′-TATACACCCATCTCCAGCGA-3′;
(R; SEQ ID NO: 9)
5′-CATCTCCTCATTCACGAAGC-3′
(Fitzsimmons et al., 1996; Marin et al., 1997).
4) Control gene-2 (SDHA):
(F; SEQ ID NO: 10)
5′-TGGGAACAAGAGGGCATCTG-3′;
(R; SEQ ID NO: 11)
5′-CCACCACTGCATCAAATTCATG-3′
(Jacob et al., 2013).

The mRNA levels were quantified using the comparative 2^{-ΔΔC(t)} method (Livak and Schmittgen, 2001).

siRNAs

All targeting and non-targeting control siRNAs were purchased as desalted duplexes and resuspended in the buffer provided by the manufacturer (Qiagen, Eurofins). The stock concentrations were adjusted to 10 and 20 μM for targeting siRNAs and non-targeting controls, respectively, and all were stored at −20 ° C. as 50 μl aliquots. The siRNA sequences used in this study were as follows (5′→3′):

(1) Control (c-siRNA):
(supplied by Eurofins.; SEQ ID NO: 12)
AGGUAGUGUAAUCGCCUUG;
(2) Neo1 (n1-siRNA):
(Guzel et al., 2019; SEQ ID NO: 13)
CUAGGCAAUUUGUCGGCUC;
(3) Neo2 (n2-siRNA):
(Guzel et al., 2019; SEQ ID NO: 14)
UAUCAUGGCGUAUGUAUCA;

Lipofectamine 2000 reagent (Invitrogen) was used as the transfection agent and the protocol was applied according to the manufacturer's instructions. Transfection of non-targeting control and targeting siRNAs were performed in parallel and a final concentration of 40 nM siRNA was achieved for each condition. Electrophysiological recordings of siRNA-treated cells were also performed in parallel at given time points and in random order. For these treatments, recordings were made from 19-37 cells from ≥4 different transfections with matching controls.

Western Blotting

Cells were washed with PBS containing 0.5 mM NaF, 0.1 mM Na₃VO₄ (Sigma-Aldrich) and lysed in RIPA buffer (0.5M Tris-HCl, pH 7.4, 1.5M NaCl, 2.5% deoxycholic acid, 10% NP-40, 10 mM EDTA) (Merck-Millipore Ltd, Watford, UK) supplemented with an EDTA-free protease inhibitor cocktail (Roche Products Ltd, Welwyn Garden City, UK). Then, cells were vortexed for 30 mins at 4° C. The lysate was centrifuged (15,000 g) at 4° C. and supernatant collected for measurement of the protein concentration by a standard Bradford assay (Bio-Rad protein assay, Bio-Rad). After 15 minutes at 70° C. in SDS loading buffer (62.5 mM Tris-HCl, 20% glycerol, 2% SDS, 100 mM DTT, 0.0025% bromphenol blue, 10% B-mercaptoethanol; pH 6.8), 50 μg of protein per lane was separated by 7.5% SDS-polyacrylamide gel electrophoresis and electroblotted onto 0.45 μm nitrocellulose membrane (Thermo Scientific). Equal protein loading was controlled by Ponceau S staining (Sigma-Aldrich). Membranes were ‘blocked’ with 5% bovine serum albumin (BSA) in TBS+0.1% Tween-20 for 1 hour and probed for 20 hours with the following primary antibodies (diluted in TBS+0.1%Tween 20+1% BSA): i) NESO-pAb antibody for nNav1.5 (1 μl/ml); and (ii) anti-actinin antibody (1 μl/ml) as loading control (Sigma-Aldrich). Secondary antibodies were horseradish peroxidase-conjugated anti-rabbit IgG for (i), and anti-mouse IgG for (ii) (Vector Laboratories Ltd, Peterborough, UK). Protein bands were visualized by enhanced chemiluminescence (Fujifilm Imaging Colorants Ltd, Manchester, UK) using Super-Signal West Dura ECL substrate (Thermo Scientific). Signal intensity of nNav1.5 was normalised to anti-actinin and averaged from 5 independent biological repeats. For each antibody, linearity of signal intensity with respect to protein concentration in the range 20-80 μg was ensured.

Immunocytochemistry

This protocol was as described previously (Fraser et al., 2005). The cells were plated on poly-L-lysine-coated (10 μg/ml) cover slips for 24-72 hours prior to brief fixation (10 min) with 4% paraformaldehyde. The primary antibody was NESOpAb, specific for nNav1.5 (Fraser et al., 2005). The secondary antibody was swine anti-rabbit conjugated to Alexafluor-568 (Invitrogen).

Cell Viability and Proliferation

Cellular viability (toxicity) and proliferation were quantified as described previously (Fraser et al., 1999, 2003b; Grimes et al., 1995). Briefly, cells were seeded into 35 mm plates at 3.5×10⁴/plate (for toxicity) or 24-well plates at 2×10⁴/well (for proliferation) and allowed to settle overnight. The cells were incubated under control conditions or treated with the drug, with a change of medium every 24 h. Cell viability was determined by trypan blue exclusion assay. Proliferation was determined by the colorimetric 3-(4,5-dimethylthiazol-2-yl)-2,5-diphenyltetrazolium bromide (MTT) assay. A ‘standard curve’ was constructed showing linearity over the experimental range. Each biological repeat (treatment and control) was performed in triplicate.

Matrigel Invasion Assay

Invasiveness was measured as described before (Fraser et al., 2005), following optimization (FIG. 8). Thus, (i) insert filters (with 8 μm pores) were coated with 50 μl of 0.21 mg/ml Matrigel (BD Biosciences, Bedford, Mass., USA); (ii) the chemotactic gradient was 0.1-10 FBS; and (iii) the cells were serum-starved for 24 hours and (iv) 10⁵ cells were seeded onto each filter. After 48 hours, the insert was swabbed and then stained with crystal violet. The invaded cells in 12 non-overlapping fields of view were counted under ×400 magnification. “Invasiveness” was calculated as the number of invaded cells normalized to the largest value observed amongst the different treatment conditions in given experimental sets. Each treatment condition was tested 5-8 times.

Data Analysis

Quantitative data were analyzed using the statistical software Origin 8.5 (OriginLab Corporation, Northampton, Mass., USA). The Shapiro-Wilk test was used to test for normality. Parametric data are presented as mean±standard error (SE); non-parametric data are presented as median and 25% and 75% interquartile ranges (data in text) and/or 5% and 95% confidence intervals (data in figures). Data were then analyzed by either Student's t-test or Mann-Whitney U-test, respectively. Proportion of cells with and without VGSCs was analyzed by the Fisher's exact test. Statistical significance is presented as: P<0.05 (*); P<0.01 (**); or P<0.001 (***).

Results

Data were obtained from CRCa cell lines mainly under normoxic conditions. Further characterizations were performed under hypoxia.

nNav1.5 mRNA and Protein Expression in CRCa Cells

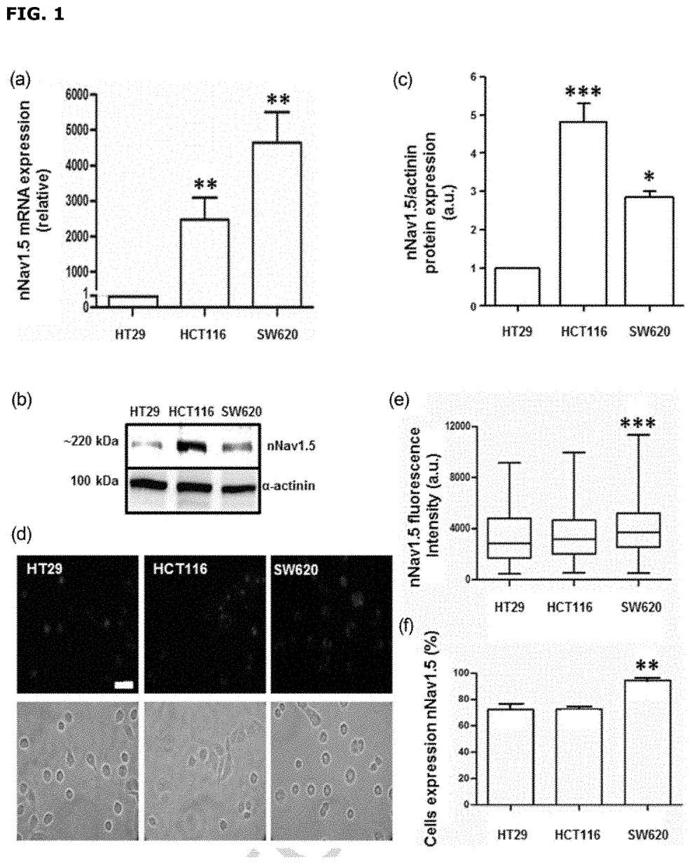

Conventional PCRs using nNav1.5-specific primers were performed on the three CRCa cell lines (FIG. 1A). The strongly metastatic human breast cancer MDA-MB-231 cells were used as positive control (data not shown; Fraser et al., 2005). nNav1.5 mRNA was detected in all three CRCa cell lines tested (FIG. 1A). Compared with HT29 cells, SW620 and HCT116 cells expressed significantly higher levels of nNav1.5 mRNA (FIG. 1A). As a confirmation, a further PCR was carried out on SW620 cells using ‘general’ Nav1.5 primers targeting the ‘spliced’ (DI:S3-S4) region (Fraser et al., 2005). When aligned, the sequence of the purified PCR product showed 99% similarity to nNav1.5 and only 88% similiarity to the ‘adult’ form (aNav1.5) (not shown). nNav1.5 protein expression in the CRCa cells was investigated using the nNav1.5-specific polyclonal antibody, NESOpAb (Chioni et al., 2005). The antibody was first revalidated for its nNav1.5 vs. aNav1.5 specificity (FIG. 9). Immunoblots showed that nNav1.5 protein (˜220 kDa) was present in all three CRCa cell lines, again SW620 and HCT116 cells expressing significantly higher levels (FIGS. 1B & C). Immunocytochemistry of non-permeabilised cells also revealed nNav1.5 protein expression in all three CRCa cell lines (FIG. 1D). There was some heterogeneity in the immunostaining but both the cell-surface fluorescence intensity and the percentage of cells stained were highest for SW620 cells (FIGS. 1E & F). It was concluded that nNav1.5 mRNA and protein expression was a feature of the three CRCa cell lines tested. For the remaining experiments, SW620 cells were adopted as a model for more detailed characterization.

Electrophysiological Effects of Differential Knock-Down of nNav1.5 and aNav1.5

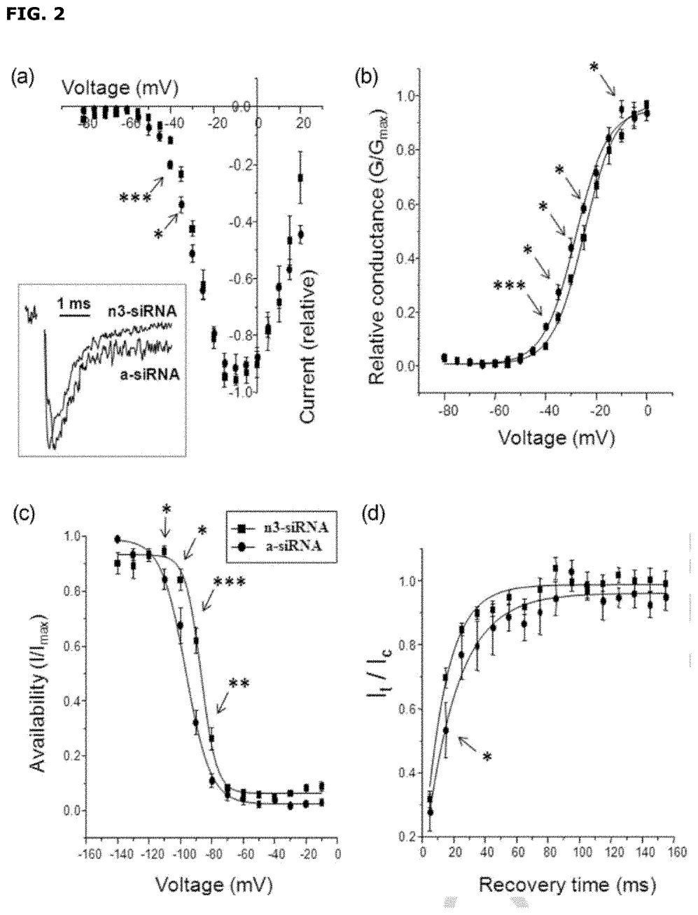

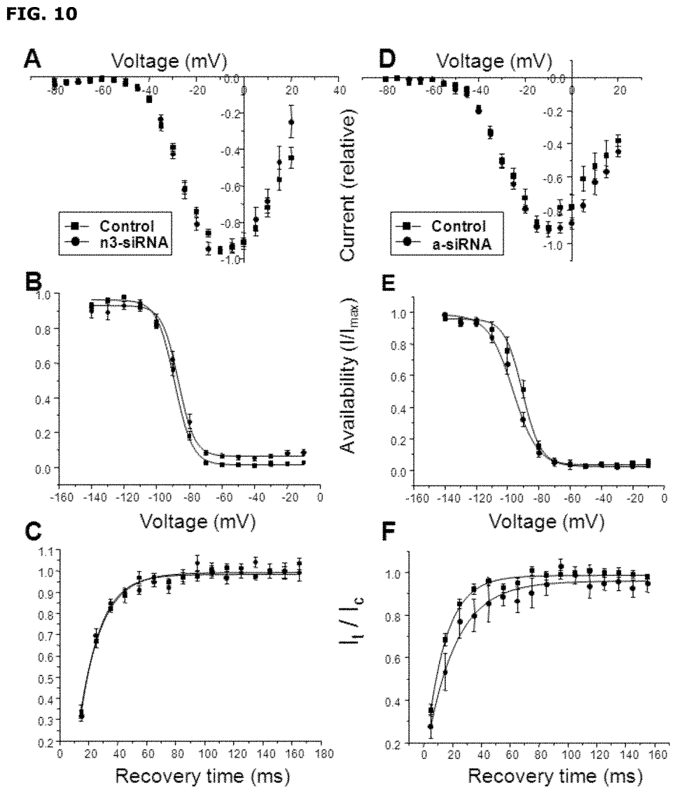

SW620 cells were transfected with siRNAs selectively targeting either nNav1.5 or aNav1.5 (with corresponding controls) and results were analysed individually and comparatively (FIG. 2; FIG. 10; Table 2).

TABLE 2
Electrophysiological effects of differential knock-down of
nNav1.5 and aNav1.5 in SW620 cells. Electrophysiological
parameters obtained from SW620 cells following differential
silencing of nNav1.5 and aNav1.5.

| Parameter | n3-siRNA | a-siRNA | N | P-value |
|---|---|---|---|---|
| Activation V_{1/2} (mV) | −24.7 ± 2.0 | −28.1 ± 1.7 | 5, 5 | <0.05 |
| Activation k (mV) | 6.1 ± 0.3 | 6.7 ± 0.6 | 5, 5 | 0.4 |
| Inactivation V_{1/2} (mV) | −86.5 ± 1.2 | −95.6 ± 2.1 | 5, 9 | <0.01 |
| Inactivation k (mV) | −5.0 ± 0.4 | −6.7 ± 0.7 | 5, 9 | <0.05 |
| T_{recovery} (ms) | 12.9 ± 0.6 | 20.9 ± 3.0 | 6, 7 | <0.05 |
| Tₚₑₐₖ (ms) | 0.66 ± 0.04 | 0.90 ± 0.06 | 10, 10 | <0.01 |
| T_{inactivation} (ms) | 0.80 ± 0.10 | 1.35 ± 0.19 | 9, 10 | <0.01 |

Abbreviations:
V_{1/2}, half-(in)activation voltage;
k, (in)activation slope factor;
T_{recovery}, recovery from inactivation time constant;
Tₚₑₐₖ, time to peak;
T_{inactivation}, inactivation time constant.
Data are shown as means ± SEMs.
For the number of measurements (n), the first and second values relate to n3-siRNA and a-siRNA, respectively.
Unpaired t-tests were used to determine statistical “P” values.

Three different siRNAs targeting the nNav1.5 sequence were used. For each siRNA, mRNA levels were compared to cells treated with control c-siRNA 90 h after transfection. Real-time RT-PCRs revealed a significant decrease in nNav1.5 mRNA levels by 50±12%, 85±10 and 32±10% for n1/n2/n3-siRNAs, respectively, in comparison to the c-siRNA (P<0.05 for all). A similar trend was observed at protein level by immunocytochemistry (not shown). Patch-clamp recordings confirmed significant reduction in peak VGSC current density for all three siRNAs: 0.0 (0-3.1) cf. 5.0 (2.2-16.8) pA/pF for n1-siRNA (P<0.001); 0.0 (0-2.4) cf. 5.3 (3.7-10.2) pA/pF for n2-siRNA (P<0.001); and 1.0 (0-6.8) cf. 8.4 (2.9-13.0) pA/pF for n3-siRNA (P<0.01), relative to the respective controls. In addition, in all cases, the proportion of cells demonstrating VGSC currents was significantly reduced: from 90 to 42% (n1-siRNA); from 91 to 33% (n2-siRNA); and from 82 to 51% (n3-siRNA) (P<0.01 cf. c-siRNA for all).

Patch-clamp recordings were also performed on SW620 cells transfected with a-siRNA targeting aNav1.5. This had noticeably less effect on the VGSC activity. Relative to controls, there was no significant reduction in peak VGSC current density: 3.5 (0-7.5) cf. 5.3 (0-10.7) pA/pF (P=0.18). Similarly, the proportion of cells expressing functional channel was not affected (58 cf. 71%; P=0.43).

We also analysed the comparative effects of the neonatal (n3) vs. adult siRNA treatments on additional characteristics of the VGSC current. These analyses demonstrated significant differences in the shifts of all tested parameters (Table 2): i) current-voltage relationships (FIG. 2A); (ii) conductance-voltage (G-V) relationships (FIG. 2B); (iii) steady-state inactivation (FIG. 2C); and (iv) recovery from inactivation (FIG. 2D). In addition, there were significant differences in the effects on half-activation voltage; half-inactivation voltage; inactivation slope factor; recovery from inactivation time constant; time to peak; and inactivation time constant (Table 2). In all cases, except one (activation V_{1/2} for a-siRNA), the directions of the shifts were as expected, i.e. n3-siRNA produced more adult-like characteristics and vice versa. The greatest differential shifts (>3-fold) were for time for peak and inactivation time constant, consistent with these parameters being characteristic of nNav1.5 (Onkal et al., 2008).

In overall conclusion, the electrophysiological data taken together confirmed that the functional VGSC expressed in SW620 cells was primarily nNav1.5.

Effects of Differential Knock-Down of nNav1.5 and aNav1.5 on Invasiveness of SW620 Cells

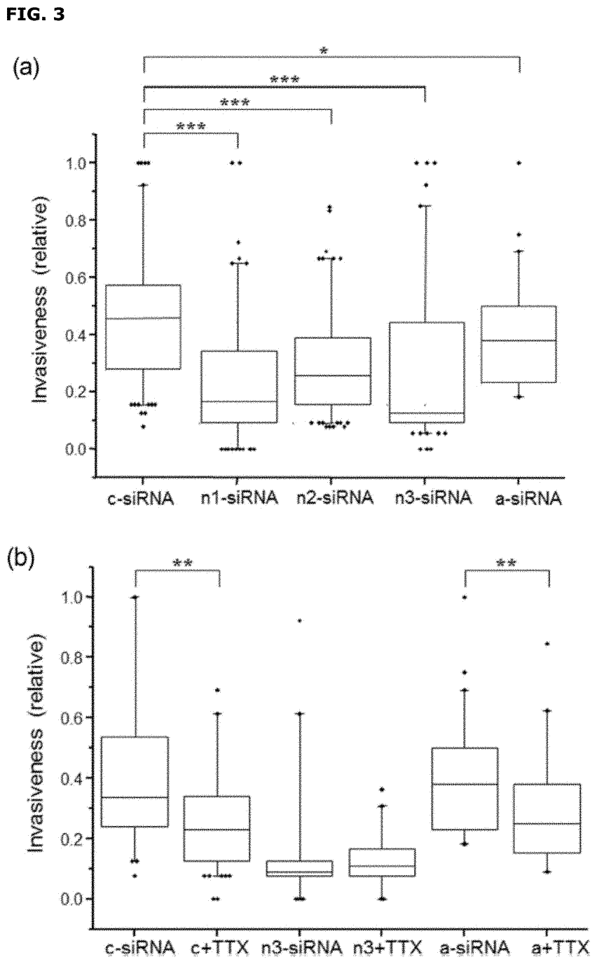

Compared to the control c-siRNA transfections, the number of invaded cells with nNav1.5 ‘silenced’ was reduced significantly by all three siRNAs: 64% (n1-siRNA), 45% (n2-siRNA), and 73% (n3-siRNA) (P<0.001 cf. c-siRNA for all) (FIG. 3A). When a-siRNA was used, there was a much smaller (17%) but significant reduction in invasion (P<0.05 cf. c-siRNA) (FIG. 3A). Importantly, subsequent treatment with TTX (20 μM) significantly reduced invasiveness of the cells treated with (i) c-siRNA by 32% or (ii) a-siRNA by 34% (FIG. 3B; P<0.01 for both). There was no difference in the effects of TTX in reducing invasion in cells transfected with c-siRNA or a-siRNA (P=0.23). In contrast, similar treatment with TTX had no effect on the cells following silencing of nNav1.5 with n3-siRNA (FIG. 3B). None of the treatment conditions had any effect on the cells' viability or proliferative activity (not shown).

These results suggested, again, that the VGSC-dependent invasiveness of SW620 cells was driven primarily by nNav1.5 activity. This agreed with the electrophysiological characterization.

Effects of Hypoxia and Ranolazine on Invasiveness

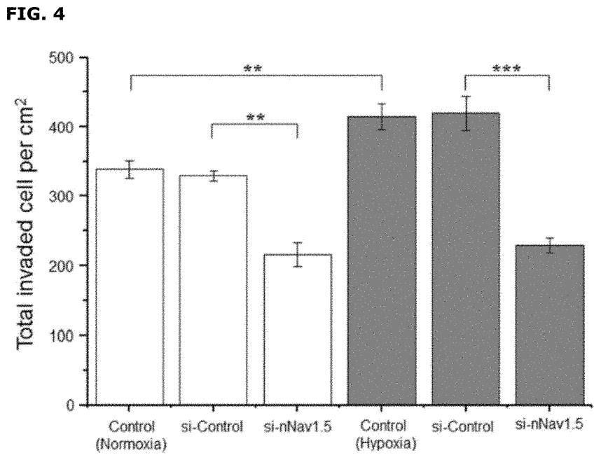

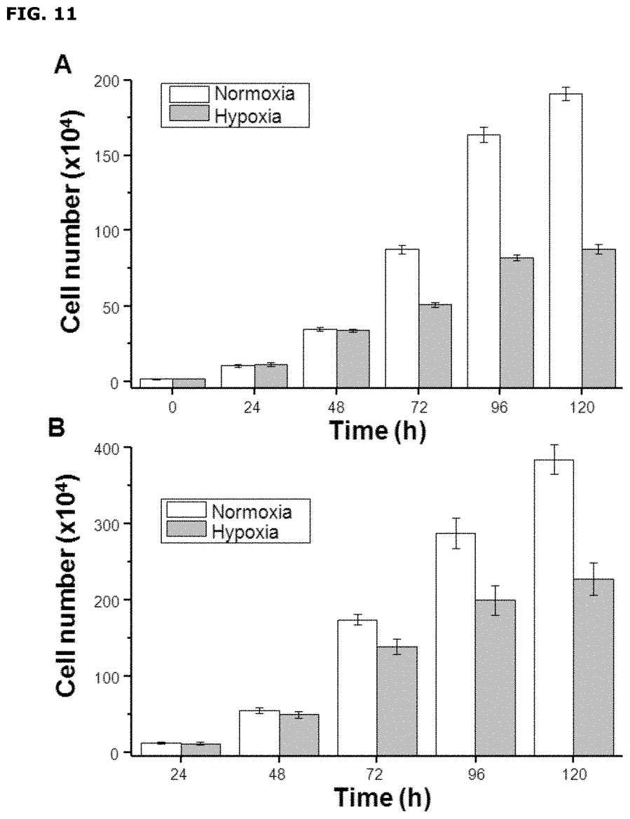

We next tested whether the predominance of nNav1.5 would prevail under hypoxia, a condition inherent to growing tumours and known generally to increase invasiveness (e.g. Krishnamachary et al., 2003). Indeed, exposing SW620 cells to hypoxia continuously for 72 h caused a significant (22%) increase in invasiveness (P<0.01 cf. normoxia; FIG. 4). c-siRNA treatments had no effect under either condition (FIG. 4). Interestingly, proliferation was significantly reduced by hypoxia treatment in a time dependent manner (FIG. 11). Thus, as determined by the MTT assay, hypoxia reduced proliferation over 72 h by 48% (P<0.05 cf. normoxia). There was no effect on cell viability (not shown). This suggested that the effect of hypoxia on invasiveness was underestimated. The hypoxia-induced increase in invasiveness was completely suppressed following treatment of the cells with n3-siRNA (FIG. 4). Importantly, the levels of cellular invasiveness attained under normoxic and hypoxic conditions with nNav1.5 silenced were the same (P=0.53). In conclusion, the hypoxia-induced increase in invasiveness was driven solely by nNav1.5 activity.

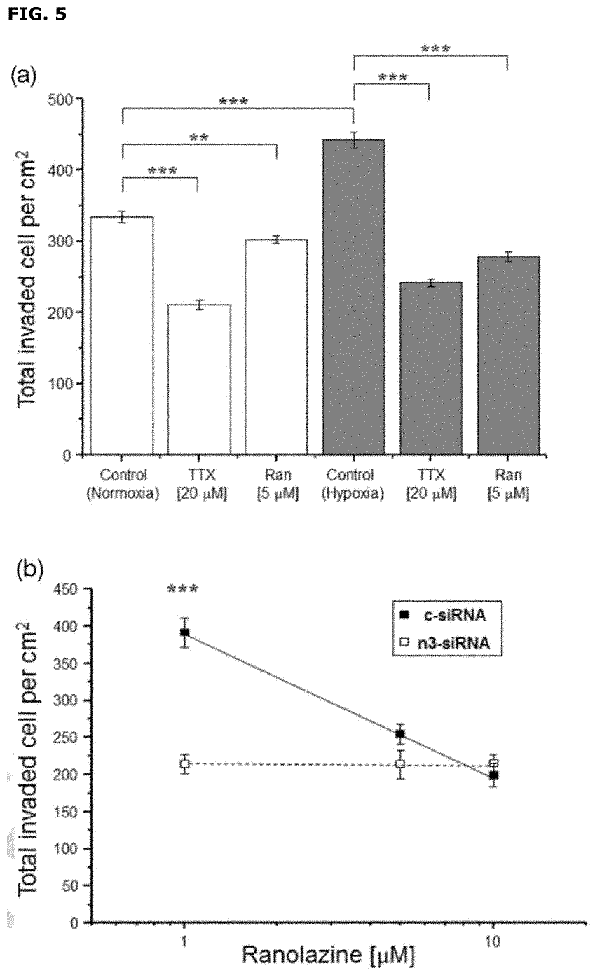

We then questioned the possible involvement in invasiveness of the channel's persistent current (I_{Nap}), itself known to be promoted by hypoxia (e.g. Ju et al., 1996). For this, we used ranolazine, a well-known blocker of I_{NaP} (e.g. Antzelevitch et al., 2004, Belardinelli et al., 2006). In this set of experiments, we first confirmed that treating the cells with TTX (20 μM) under normoxic conditions caused inhibition of invasiveness (by 37%; P<0.001 cf. control; FIG. 5A). As expected, lowering the concentration of TTX to 1 μM had no effect (not shown). As before, exposing the cells to hypoxia for 72 h caused a significant increase in invasiveness (by 33%; P<0.001 cf. normoxia); TTX (20 μM) reduced it by 45% (P<0.001 cf. hypoxia control; FIG. 5A). Under normoxia, ranolazine (5 μM) caused only a small (9%) but significant reduction in invasion (P<0.01 cf. control; FIG. 5A). Under hypoxia, however, the effect of 5 μM ranolazine was significantly increased to 37% (P<0.001 cf. both 5 μM ranolazine under normoxia, and the hypoxia control). Proliferation was not affected by 20 μM TTX or up to 10 μM ranolazine under normoxia or hypoxia (not shown).

We also tested whether the effect of ranolazine under hypoxia was mediated by nNav1.5. In control/c-siRNA-treated cells, ranolazine inhibited invasion in a dose-dependent manner with two-fold difference over 1-10 μM (FIG. 5B). In cells pre-treated with n3-siRNA, however, invasiveness was suppressed, as shown before, and ranolazine had no additional effect even at the highest concentration (10 μM) used (FIG. 5B).

It was concluded (i) that hypoxia promoted invasiveness via nNav1.5 activity and (ii) that ranolazine blocked this effect.

Effects on nNav1.5 mRNA and Protein Expression

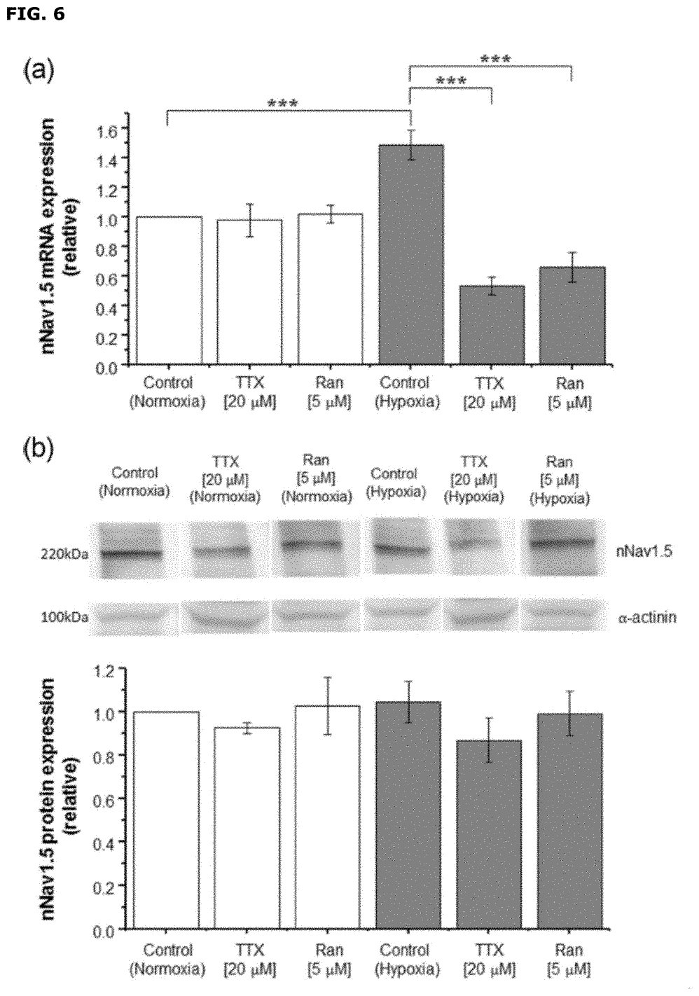

Finally, we questioned whether nNav1.5 mRNA and protein expression would change under hypoxia. Under normoxic conditions, neither TTX (20 μM) nor ranolazine (5 μM) had any effect on the mRNA expression (FIG. 6A). On the other hand, exposing the cells to hypoxia for 48 h increased nNav1.5 mRNA expression by 49% (P<0.01) and both TTX and ranolazine blocked this increase. In contrast, hypoxia had no effect on nNav1.5 protein expression and TTX and ranolazine also had no effect (FIG. 6B).

Discussion

The main results were as follows: 1) nNav1.5 mRNA and protein were expressed commonly in all three CRCa (HT29, HCT116 and SW620) cell lines tested. Expression levels were generally higher in the SW620 cells and comparable to the MDA-MB-231 cells also expressing nNav1.5 functionally. (2) Electrophysiology revealed that silencing nNav1.5 caused shifts towards aNav1.5-like characteristics and vice versa. However, the effect of silencing nNav1.5 was significantly greater. (3) Three different siRNAs suppressing nNav1.5 expression in SW620 cells all reduced Matrigel invasiveness. (4) TTX had no effect on invasiveness of cells pre-treated with siRNA targeting nNav1.5. (5) Silencing aNav1.5 also caused an apparent inhibition of invasiveness. In contrast to nNav1.5, however, TTX still significantly reduced invasiveness in cells treated with aNav1.5 siRNA. (6) Hypoxia increased cellular invasiveness. This effect was lost in cells treated with nNav1.5 siRNA. (7) Ranolazine significantly reduced invasiveness much more under hypoxia than normoxia but had no effect on invasiveness in hypoxic cells pre-treated with siRNA targeting nNav1.5. (8) During hypoxia, nNav1.5 mRNA expression increased (blocked by ranolazine) but there was no effect at protein level.

Prevalence of nNav1.5 Expression in CRCa Cells

Conventional PCRs performed with specific primers detected nNav1.5 mRNAs in all three CRCa cell lines tested. Primers targeting the developmentally regulated DI:S3-S4 region of Nav1.5 and sequencing the PCR products confirmed that nNav1.5 mRNA was present. A polyclonal antibody, NESOpAb, that specifically recognizes nNav1.5 protein with high selectivity over its ‘nearest neighbour’, aNav1.5, was produced earlier (Chioni et al., 2005) and revalidated. Furthermore, NESOpAb would bind to an external epitope thus enabling expression in plasma membrane to be assessed in non-permeabilized cells. Westerns blots and immunocytochemistry using NESOpAb showed that nNav1.5 protein was also present in all cell lines. Expression was generally higher and more consistent in the metastatic/poorly differentiated SW620 and HCT116 cell lines compared with the relatively differentiated HT29 cells. This was especially so for the immunocytochemistry data which showed higher levels of nNav1.5 protein in the plasma membrane, where the channel would be functional. However, some inconsistency was noted between the mRNA/protein levels in the cell lines. Such mismatch between mRNA/protein levels has been reported previously, including in cancer cells (e.g. Tian et al., 2004; Zhang et al., 2014). Overall, these results extend the results of House et al. (2010) and Baptista-Hon et al. (2014). In human breast cancer cells also, nNav1.5 was found to be expressed and dominant (Brackenbury et al., 2007; Fraser et al., 2005). The expression of a ‘neonatal’ splice variant fits well with the phenomenon of embryonic gene expression in cancer (e.g. Ben-Porath et al., 2008).

Treatments of SW620 cells with three different siRNAs targeting nNav1.5 (n1/2/3-siRNAs) all resulted in significant reduction of nNav1.5 mRNA levels, compared with the control treatment (c-siRNA). Concurrently, both the proportion of cells expressing functional nNav1.5 and the associated current density were reduced significantly. In contrast, the quantitative effects of silencing aNav1.5 were significantly less. Comparing the residual inward currents in cells treated with n3-siRNA vs. a-siRNA revealed significant differences in the shifts of the following characteristics: i) current-voltage relationship; (ii) voltage dependence of conductance; (iii) steady-state inactivation; and (iv) recovery from inactivation. In addition, there were significant differences for half-activation voltage, half-inactivation voltage, inactivation slope factor, recovery from inactivation time constant, time to peak, and the inactivation time constant. These effects agree generally with the reported differential characteristics of nNav1.5 and aNav1.5 (Onkal et al., 2008). Taken together, these analyses suggested that nNav1.5 made by far the greatest contribution to the VGSC current in the SW620 cells.

Control of Invasiveness Predominantly by nNav1.5

The three siRNA treatments targeting nNav1.5 suppressed invasiveness of the SW620 cells by 45-73%, comparable to the effect of TTX. Indeed, when the n3-siRNA transfected cells were additionally treated with TTX, there was no further decrease in invasion. In agreement with the electrophysiology, therefore, it was concluded that nNav1.5 was predominant in controlling the VGSC-dependent component of invasiveness. However, a-siRNA also had an inhibitory effect on invasiveness, consistent with the presence of some functional aNav1.5 as seen in the differential electrophysiological effects of a-siRNA vs. n3-siRNA (FIG. 3). The size of the a-siRNA effect on invasiveness (17%) was rather surprising considering that the corresponding inhibition of current density (as well as the percentage of cells expressing functional channel) was, in fact, non-significant. This could indicate that the relationship between VGSC expression/activity and its contribution to invasiveness is steep (Djamgoz, 2011; see also Brackenbury et al., 2007).

Interestingly, the specific neonatal nature of the VGSC may not be essential for its contribution to invasiveness. Such a splice variant could just be a by-product of the overall dedifferentiation process inherent to cancer. Even the subtype of VGSC expressed may not be significant, reflecting merely tissue specificity of expression. This point was demonstrated directly in a study on non-metastatic prostate cancer cells in which overexpression of a ‘non-dominant’ VGSC (Nav1.4 rather than the dominant Nav1.7) was found to be “necessary and sufficient” for invasiveness (Bennett et al., 2004). Instead, it may be the influx of Na⁺ that the channels mediate that is important (Brackenbury and Djamgoz, 2006). This agrees with previous work showing that VGSC ‘openers’ (e.g. aconitine and veratridine) increase metastatic cell behaviours, including invasiveness (Fraser et al., 2003b, 2005; House et al., 2015). Furthermore, the Na⁺ content of tumour cells and tissues is known generally to be higher than normal tissues (e.g. Ouwerkerk et al., 2007; Roger et al., 2007).

Finally, the pathway(s) through which nNav1.5 activity enhances invasiveness warrants further study. House et al. (2010) revealed SCN5A (the gene encoding Nav1.5) to be an upstream “key regulator” of a network of genes including those for Ca²⁺ signalling, MAP kinase and proteases. In addition, evidence from breast cancer, where nNav1.5 has been more extensively studied, has shown channel activity to be linked to pericellular acidification and activation of cathepsin B and MMP9 (Gillet et al., 2009; Nelson et al., 2015). For CRCa, it may be worth exploring the link between nNav1.5 activity and MMP7 expression, since earlier research has shown positive correlation between increased MMP7 expression and tumor invasion in HCT116 and SW620 cell lines compared to the HT29 cell line (Banskota et al., 2015).

Hypoxia-Induced Increase in Invasiveness: Control by nNav1.5 and Inhibition by Ranolazine

The effects of hypoxia on CRCa cell behaviour appear to be cell-type dependent (Tatrai et al., 2017). As regards invasiveness, most evidence indicates an enhancement (e.g. Hongo et al., 2013). This was also the case here. Thus, exposing SW620 cells to hypoxia significantly increased their invasiveness. It is of interest to note that normal colorectal tissue may only be at 6.8% O₂ and CRCa cells are exposed to 2-4% O₂ (Mckeown, 2014). It could be argued, therefore, that the ‘hypoxia’ induced by 1% O₂ in our experiments might actually correspond to the basal tumour condition whilst the ‘atmospheric’ (ca. 20%) O₂ in the normal cultures represents ‘hyperoxygenation’. Nevertheless, subjecting the SW620 cells to relative hypoxia (i.e. ˜20 to 1%) enhanced their invasiveness and this was blocked completely by pre-treatment with n3-siRNA suggesting that it was nNav1.5 that was the underlying VGSC. Thus, the predominance of nNav1.5 in controlling SW620 invasiveness that was demonstrated under normoxia was maintained under hypoxia.

The effect of hypoxia on the invasiveness of SW620 cells was also suppressed by ranolazine. Thus, treatment of the cells with 5 μM ranolazine inhibited invasion significantly by 37%. Importantly, this effect of ranolazine was lost after silencing nNav1.5 demonstrating that the key role played by nNav1.5 in promoting invasiveness manifests itself also in pharmacological control. Ranolazine has been shown independently to block the hypoxia-induced persistent current, I_{NaP}, of VGSC, especially Nav1.5, with an IC₅₀ of 5.9 μM (Antzelevitch et al., 2004). Taken together, our results are consistent with I_{NaP} mediating the hypoxia-induced increase in invasiveness. Under normoxia, however, the effect of ranolazine was much smaller than the effect of TTX (9 vs. 37%). This would imply that the transient current (I_{NaT}) of the VGSC made the significant contribution to invasiveness. More research may elucidate the differential pro-invasive roles of these separate current components under normoxia vs. hypoxia. Interestingly, hypoxia concurrently promoted nNav1.5 mRNA (but not protein expression) and this was also inhibited by ranolazine. Without being limited to theory, it would appear, therefore, that further effects of hypoxia may occur in the longer term.

### Conclusions

Our results show that nNav1.5 is a viable functional biomarker and target for managing invasive CRCa and other cancers associated with nNav1.5 expression, particularly using gene therapeutic approaches. As suggested initially by House et al. (2010), nNav1.5 is expressed early in metastasis and is upstream of several canonical signalling mechanisms of invasiveness, consistent with its pathophysiology. Approximately 50% of CRCa patients relapse, some with distant metastases, even after surgery and/or chemotherapy and, thus, earlier diagnosis is vital (Young et al., 2014). Second, as a neonatal splice variant, it may be ‘cancer specific’ in the adult body and can be targeted with gene therapy or antibody, both for diagnosis and therapy (Chioni et al., 2005; Brackenbury et al., 2007; Yamaci et al., 2017). In this light, a recent study has reported that high Nav1.5 expression levels correlated with unfavourable disease-free survival in patients with non-metastatic CRCa (Peng et al., 2017). Moreover, ranolazine has been shown in this study to reduce CRCa cell invasiveness at clinical doses (<10 μM), and was found previously to reduce metastatic dissemination in a breast cancer xenograft model (Driffort et al., 2014). See also Djamgoz and Onkal, 2013; and Koltai, 2015; Nelson et al., 2015

SEQUENCE TABLE

| SEQ ID NO/ | |
|---|---|
| Description | Sequence |
| SEQ ID NO: 1 | MANFLLPRGTSSFRRFTRESLAAIEKRMAEKQARGSTTLQESREGLPEEEAP |
| aNav1.5 | RPQLDLQASKKLPDLYGNPPQELIGEPLEDLDPFYSTQKTFIVLNKGKTIFR |
| UniProtKB- | FSATNALYVLSPFHPIRRAAVKILVHSLFNMLIMCTILTNCVFMAQHDPPPW |
| Q14524 | TKYVEYTFTAIYTFESLVKILARGFCLHAFTFLRDPWNWLDFSVIIMAYTTE |
| (SCN5A_HUMAN), | FVDLGNVSALRTFRVLRALKTISVISGLKTIVGALIQSVKKLADVMVLTVFC |
| ISOFORM-1 | LSVFALIGLQLFMGNLRHKCVRNFTALNGTNGSVEADGLVWESLDLYLSDPE |
| (Nav1.5c) | NYLLKNGTSDVLLCGNSSDAGTCPEGYRCLKAGENPDHGYTSFDSFAWAFLA |
| >sp\|Q14524\| | LFRLMTQDCWERLYQQTLRSAGKIYMIFFMLVIFLGSFYLVNLILAVVAMAY |
| SCN5A_HUMAN | EEQNQATIAETEEKEKRFQEAMEMLKKEHEALTIRGVDTVSRSSLEMSPLAP |
| Sodium channel | VNSHERRSKRRKRMSSGTEECGEDRLPKSDSEDGPRAMNHLSLTRGLSRTSM |
| protein type 5 | KPRSSRGSIFTFRRRDLGSEADFADDENSTAGESESHHTSLLVPWPLRRTSA |
| subunit alpha | QGQPSPGTSAPGHALHGKKNSTVDCNGVVSLLGAGDPEATSPGSHLLRPVML |
| OS = Homo sapiens | EHPPDTTTPSEEPGGPQMLTSQAPCVDGFEEPGARQRALSAVSVLTSALEEL |
| OX = 9606 | EESRHKCPPCWNRLAQRYLIWECCPLWMSIKQGVKLVVMDPFTDLTITMCIV |
| GN = SCN5A PE = 1 | LNTLFMALEHYNMTSEFEEMLQVGNLVFTGIFTAEMTFKIIALDPYYYFQQG |
| SV = 2 | WNIFDSIIVILSLMELGLSRMSNLSVLRSFRLLRVFKLAKSWPTLNTLIKII |
| | GNSVGALGNLTLVLAIIVFIFAVVGMQLFGKNYSELRDSDSGLLPRWHMMDF |
| | FHAFLIIFRILCGEWIETMWDCMEVSGQSLCLLVFLLVMVIGNLVVLNLFLA |
| | LLLSSFSADNLTAPDEDREMNNLQLALARIQRGLRFVKRTTWDFCCGLLRQR |
| | PQKPAALAAQGQLPSCIATPYSPPPPETEKVPPTRKETRFEEGEQPGQGTPG |
| | DPEPVCVPIAVAESDTDDQEEDEENSLGTEEESSKQQESQPVSGGPEAPPDS |
| | RTWSQVSATASSEAEASASQADWRQQWKAEPQAPGCGETPEDSCSEGSTADM |
| | TNTAELLEQIPDLGQDVKDPEDCFTEGCVRRCPCCAVDTTQAPGKVWWRLRK |
| | TCYHIVEHSWFETFIIFMILLSSGALAFEDIYLEERKTIKVLLEYADKMFTY |
| | VFVLEMLLKWVAYGFKKYFTNAWCWLDFLIVDVSLVSLVANTLGFAEMGPIK |
| | SLRTLRALRPLRALSRFEGMRVVVNALVGAIPSIMNVLLVCLIFWLIFSIMG |
| | VNLFAGKFGRCINQTEGDLPLNYTIVNNKSQCESLNLTGELYWTKVKVNFDN |
| | VGAGYLALLQVATFKGWMDIMYAAVDSRGYEEQPQWEYNLYMYIYFVIFIIF |
| | GSFFTLNLFIGVIIDNFNQQKKKLGGQDIFMTEEQKKYYNAMKKLGSKKPQK |
| | PIPRPLNKYQGFIFDIVTKQAFDVTIMFLICLNMVTMMVETDDQSPEKINIL |
| | AKINLLFVAIFTGECIVKLAALRHYYFTNSWNIFDFVVVILSIVGTVLSDII |
| | QKYFFSPTLFRVIRLARIGRILRLIRGAKGIRTLLFALMMSLPALFNIGLLL |
| | FLVMFIYSIFGMANFAYVKWEAGIDDMFNFQTFANSMLCLFQITTSAGWDGL |
| | LSPILNTGPPYCDPTLPNSNGSRGDCGSPAVGILFFTTYIIISFLIVVNMYI |
| | AIILENFSVATEESTEPLSEDDFDMFYEIWEKFDPEATQFIEYSVLSDFADA |
| | LSEPLRIAKPNQISLINMDLPMVSGDRIHCMDILFAFTKRVLGESGEMDALK |
| | IQMEEKFMAANPSKISYEPITTTLRRKHEEVSAMVIQRAFRRHLLQRSLKHA |
| | SFLFRQQAGSGLSEEDAPEREGLIAYVMSENFSRPLGPPSSSSISSTSFPPS |
| | YDSVTRATSDNLQVRGSDYSHSEDLADFPPSPDRDRESIV |
| SEQ ID NO: 2 | agacggcggcggcgcccgtaggatgcagggatcgctcccccggggccgctga |
| >Homo sapiens | gcctgcgcccagtgccccgagccccgcgccgagccgagtccgcgccaagcag |
| sodium voltage- | cagccgcccaccccggggcccggccgggggaccagcagcttccccacaggca |
| gated channel | acgtgaggagagcctgtgcccagaagcaggatgagaagatggcaaacttcct |
| alpha subunit 5 | attacctcggggcaccagcagcttccgcaggttcacacgggagtccctggca |
| (SCN5A), | gccatcgagaagcgcatggcagagaagcaagcccgcggctcaaccaccttgc |
| transcript variant | aggagagccgagaggggctgcccgaggaggaggctccccggccccagctgga |
| 1, mRNA. NCBI | cctgcaggcctccaaaaagctgccagatctctatggcaatccaccccaagag |
| Reference Sequence: | ctcatcggagagcccctggaggacctggaccccttctatagcacccaaaaga |
| NM_198056.2GenBank | ctttcatcgtactgaataaaggcaagaccatcttccggttcagtgccaccaa |
| Graphics | cgccttgtatgtcctcagtcccttccaccccatccggagagcggctgtgaag |
| >NM_198056.2 | attctggttcactcgctcttcaacatgctcatcatgtgcaccatcctcacca |
| Homo sapiens | actgcgtgttcatggcccagcacgaccctccaccctggaccaagtatgtcga |
| sodium voltage- | gtacaccttcaccgccatttacacctttgagtctctggtcaagattctggct |
| gated channel | cgaggcttctgcctgcacgcgttcactttccttcgggacccatggaactggc |
| alpha subunit 5 | tggactttagtgtgattatcatggcatacacaactgaatttgtggacctggg |
| (SCN5A), | caatgtctcagccttacgcaccttccgagtcctccgggccctgaaaactata |
| transcript variant | tcagtcatttcagggctgaagaccatcgtgggggccctgatccagtctgtga |
| 1, mRNA | agaagctggctgatgtgatggtcctcacagtcttctgcctcagcgtctttgc |
| [mRNA format is | cctcatcggcctgcagctcttcatgggcaacctaaggcacaagtgcgtgcgc |
| obtained by | aacttcacagcgctcaacggcaccaacggctccgtggaggccgacggcttgg |
| exchanging | tctgggaatccctggacctttacctcagtgatccagaaaattacctgctcaa |
| thymine (T) for | gaacggcacctctgatgtgttactgtgtgggaacagctctgacgctgggaca |
| uracil (U)] | tgtccggagggctaccggtgcctaaaggcaggcgagaaccccgaccacggct |
| | acaccagcttcgattcctttgcctgggcctttcttgcactcttccgcctgat |
| | gacgcaggactgctgggagcgcctctatcagcagaccctcaggtccgcaggg |
| | aagatctacatgatcttcttcatgcttgtcatcttcctggggtccttctacc |
| | tggtgaacctgatcctggccgtggtcgcaatggcctatgaggagcaaaacca |
| | agccaccatcgctgagaccgaggagaaggaaaagcgcttccaggaggccatg |
| | gaaatgctcaagaaagaacacgaggccctcaccatcaggggtgtggataccg |
| | tgtcccgtagctccttggagatgtcccctttggccccagtaaacagccatga |
| | gagaagaagcaagaggagaaaacggatgtcttcaggaactgaggagtgtggg |
| | gaggacaggctccccaagtctgactcagaagatggtcccagagcaatgaatc |
| | atctcagcctcacccgtggcctcagcaggacttctatgaagccacgttccag |
| | ccgcgggagcattttcacctttcgcaggcgagacctgggttctgaagcagat |
| | tttgcagatgatgaaaacagcacagcgggggagagcgagagccaccacacat |
| | cactgctggtgccctggcccctgcgccggaccagtgcccagggacagcccag |
| | tcccggaacctcggctcctggccacgccctccatggcaaaaagaacagcact |
| | gtggactgcaatggggtggtctcattactgggggcaggcgacccagaggcca |
| | catccccaggaagccacctcctccgccctgtgatgctagagcacccgccaga |
| | cacgaccacgccatcggaggagccaggcgggccccagatgctgacctcccag |
| | gctccgtgtgtagatggcttcgaggagccaggagcacggcagcgggccctca |
| | gcgcagtcagcgtcctcaccagcgcactggaagagttagaggagtctcgcca |
| | caagtgtccaccatgctggaaccgtctcgcccagcgctacctgatctgggag |
| | tgctgcccgctgtggatgtccatcaagcagggagtgaagttggtggtcatgg |
| | acccgtttactgacctcaccatcactatgtgcatcgtactcaacacactctt |
| | catggcgctggagcactacaacatgacaagtgaattcgaggagatgctgcag |
| | gtcggaaacctggtcttcacagggattttcacagcagagatgaccttcaaga |
| | tcattgccctcgacccctactactacttccaacagggctggaacatcttcga |
| | cagcatcatcgtcatccttagcctcatggagctgggcctgtcccgcatgagc |
| | aacttgtcggtgctgcgctccttccgcctgctgcgggtcttcaagctggcca |
| | aatcatggcccaccctgaacacactcatcaagatcatcgggaactcagtggg |
| | ggcactggggaacctgacactggtgctagccatcatcgtgttcatctttgct |
| | gtggtgggcatgcagctctttggcaagaactactcggagctgagggacagcg |
| | actcaggcctgctgcctcgctggcacatgatggacttctttcatgccttcct |
| | catcatcttccgcatcctctgtggagagtggatcgagaccatgtgggactgc |
| | atggaggtgtcggggcagtcattatgcctgctggtcttcttgcttgttatgg |
| | tcattggcaaccttgtggtcctgaatctcttcctggccttgctgctcagctc |
| | cttcagtgcagacaacctcacagcccctgatgaggacagagagatgaacaac |
| | ctccagctggccctggcccgcatccagaggggcctgcgctttgtcaagcgga |
| | ccacctgggatttctgctgtggtctcctgcggcagcggcctcagaagcccgc |
| | agcccttgccgcccagggccagctgcccagctgcattgccaccccctactcc |
| | ccgccacccccagagacggagaaggtgcctcccacccgcaaggaaacacggt |
| | ttgaggaaggcgagcaaccaggccagggcacccccggggatccagagcccgt |
| | gtgtgtgcccatcgctgtggccgagtcagacacagatgaccaagaagaagat |
| | gaggagaacagcctgggcacggaggaggagtccagcaagcagcaggaatccc |
| | agcctgtgtccggtggcccagaggcccctccggattccaggacctggagcca |
| | ggtgtcagcgactgcctcctctgaggccgaggccagtgcatctcaggccgac |
| | tggcggcagcagtggaaagcggaaccccaggccccagggtgcggtgagaccc |
| | cagaggacagttgctccgagggcagcacagcagacatgaccaacaccgctga |
| | gctcctggagcagatccctgacctcggccaggatgtcaaggacccagaggac |
| | tgcttcactgaaggctgtgtccggcgctgtccctgctgtgcggtggacacca |
| | cacaggccccagggaaggtctggtggcggttgcgcaagacctgctaccacat |
| | cgtggagcacagctggttcgagacattcatcatcttcatgatcctactcagc |
| | agtggagcgctggccttcgaggacatctacctagaggagcggaagaccatca |
| | aggttctgcttgagtatgccgacaagatgttcacatatgtcttcgtgctgga |
| | gatgctgctcaagtgggtggcctacggcttcaagaagtacttcaccaatgcc |
| | tggtgctggctcgacttcctcatcgtagacgtctctctggtcagcctggtgg |
| | ccaacaccctgggctttgccgagatgggccccatcaagtcactgcggacgct |
| | gcgtgcactccgtcctctgagagctctgtcacgatttgagggcatgagggtg |
| | gtggtcaatgccctggtgggcgccatcccgtccatcatgaacgtcctcctcg |
| | tctgcctcatcttctggctcatcttcagcatcatgggcgtgaacctctttgc |
| | ggggaagtttgggaggtgcatcaaccagacagagggagacttgcctttgaac |
| | tacaccatcgtgaacaacaagagccagtgtgagtccttgaacttgaccggag |
| | aattgtactggaccaaggtgaaagtcaactttgacaacgtgggggccgggta |
| | cctggcccttctgcaggtggcaacatttaaaggctggatggacattatgtat |
| | gcagctgtggactccagggggtatgaagagcagcctcagtgggaatacaacc |
| | tctacatgtacatctattttgtcattttcatcatctttgggtctttcttcac |
| | cctgaacctctttattggtgtcatcattgacaacttcaaccaacagaagaaa |
| | aagttagggggccaggacatcttcatgacagaggagcagaagaagtactaca |
| | atgccatgaagaagctgggctccaagaagccccagaagcccatcccacggcc |
| | cctgaacaagtaccagggcttcatattcgacattgtgaccaagcaggccttt |
| | gacgtcaccatcatgtttctgatctgcttgaatatggtgaccatgatggtgg |
| | agacagatgaccaaagtcctgagaaaatcaacatcttggccaagatcaacct |
| | gctctttgtggccatcttcacaggcgagtgtattgtcaagctggctgccctg |
| | cgccactactacttcaccaacagctggaatatcttcgacttcgtggttgtca |
| | tcctctccatcgtgggcactgtgctctcggacatcatccagaagtacttctt |
| | ctccccgacgctcttccgagtcatccgcctggcccgaataggccgcatcctc |
| | agactgatccgaggggccaaggggatccgcacgctgctctttgccctcatga |
| | tgtccctgcctgccctcttcaacatcgggctgctgctcttcctcgtcatgtt |
| | catctactccatctttggcatggccaacttcgcttatgtcaagtgggaggct |
| | ggcatcgacgacatgttcaacttccagaccttcgccaacagcatgctgtgcc |
| | tcttccagatcaccacgtcggccggctgggatggcctcctcagccccatcct |
| | caacactgggccgccctactgcgaccccactctgcccaacagcaatggctct |
| | cggggggactgcgggagcccagccgtgggcatcctcttcttcaccacctaca |
| | tcatcatctccttcctcatcgtggtcaacatgtacattgccatcatcctgga |
| | gaacttcagcgtggccacggaggagagcaccgagcccctgagtgaggacgac |
| | ttcgatatgttctatgagatctgggagaaatttgacccagaggccactcagt |
| | ttattgagtattcggtcctgtctgactttgccgatgccctgtctgagccact |
| | ccgtatcgccaagcccaaccagataagcctcatcaacatggacctgcccatg |
| | gtgagtggggaccgcatccattgcatggacattctctttgccttcaccaaaa |
| | gggtcctgggggagtctggggagatggacgccctgaagatccagatggagga |
| | gaagttcatggcagccaacccatccaagatctcctacgagcccatcaccacc |
| | acactccggcgcaagcacgaagaggtgtcggccatggttatccagagagcct |
| | tccgcaggcacctgctgcaacgctctttgaagcatgcctccttcctcttccg |
| | tcagcaggcgggcagcggcctctccgaagaggatgcccctgagcgagagggc |
| | ctcatcgcctacgtgatgagtgagaacttctcccgaccccttggcccaccct |
| | ccagctcctccatctcctccacttccttcccaccctcctatgacagtgtcac |
| | tagagccaccagcgataacctccaggtgcgggggtctgactacagccacagt |
| | gaagatctcgccgacttccccccttctccggacagggaccgtgagtccatcg |
| | tgtgagcctcggcctggctggccaggacacactgaaaagcagcctttttcac |
| | catggcaaacctaaatgcagtcagtcacaaaccagcctggggccttcctggc |
| | tttgggagtaagaaatgggcctcagccccgcggatcaaccaggcagagttct |
| | gtggcgccgcgtggacagccggagcagttggcctgtgcttggaggcctcaga |
| | tagacctgtgacctggtctggtcaggcaatgccctgcggctctggaaagcaa |
| | cttcatcccagctgctgaggcgaaatataaaactgagactgtatatgttgtg |
| | aatgggctttcataaatttattatatttgatatttttttacttgagcaaaga |
| | actaaggatttttccatggacatgggcagcaattcacgctgtctcttcttaa |
| | ccctgaacaagagtgtctatggagcagccggaagtctgttctcaaagcagaa |
| | gtggaatccagtgtggctcccacaggtcttcactgcccaggggtcgaatggg |
| | gtccccctcccacttgacctgagatgctgggagggctgaacccccactcaca |
| | caagcacacacacacagtcctcacacacggaggccagacacaggccgtggga |
| | cccaggctcccagcctaagggagacaggcctttccctgccggccccccaagg |
| | atggggttcttgtccacggggctcactctggccccctattgtctccaaggtc |
| | ccattttccccctgtgttttcacgcaggtcatattgtcagtcctacaaaaat |
| | aaaaggcttccagaggagagtggcctgggtcccagggctggccctaggcact |
| | gatagttgccttttcttcccctcctgtaagagtattaacaaaaccaaaggac |
| | acaagggtgcaagccccattcacggcctggcatgcagcttgtccttgctcct |
| | ggaacctggcaggccctgcccagccagccatcggaagagagggctgagccat |
| | gggggtttggggctaagaagttcaccagccctgagccatggcggcccctcag |
| | cctgcctgaagagaggaaactggcgatctcccagggctctctggaccatacg |
| | cggaggagttttctgtgtggtctccagctcctctccagacacagagacatgg |
| | gagtggggagcggagcttggccctgcgccctgtgcagggaaagggatggtca |
| | ggcccagttctcgtgcccttagaggggaatgaaccatggcacctttgagaga |
| | gggggcactgtggtcaggcccagcctctctggctcagcccgggatcctgatg |
| | gcacccacacagaggacctctttggggcaagatccaggtggtcccataggtc |
| | ttgtgaaaaggctttttcagggaaaaatattttactagtccaatcaccccca |
| | ggacctcttcagctgctgacaatcctatttagcatatgcaaatcttttaaca |
| | tagagaactgtcaccctgaggtaacagggtcaactggcgaagcctgagcagg |
| | caggggcttggctgccccattccagctctcccatggagcccctccaccgggc |
| | gcatgcctcccaggccacctcagtctcacctgccggctctgggctggctgct |
| | cctaacctacctcgccgagctgtcggagggctggacatttgtggcagtgctg |
| | aagggggcattgccggcgagtaaagtattatgtttcttcttgtcaccccagt |
| | tcccttggtggcaaccccagacccaacccatgcccctgacagatctagttct |
| | cttctcctgtgttccctttgagtccagtgtgggacacggtttaactgtccca |
| | gcgacatttctccaagtggaaatcctatttttgtagatctccatgctttgct |
| | ctcaaggcttggagaggtatgtgcccctcctgggtgctcaccgcctgctaca |
| | caggcaggaatgcggttgggaggcaggtcgggctgccagcccagctggccgg |
| | aaggagactgtggtttttgtgtgtgtggacagcccgggagctttgagacagg |
| | tgcctggggctggctgcagacggtgtggttgggggtgggaggtgagctagac |
| | ccaacccttagcttttagcctggctgtcacctttttaatttccagaactgca |
| | caatgaccagcaggagggaaggacagacatcaagtgccagatgttgtctgaa |
| | ctaatcgagcacttctcaccaaacttcatgtataaataaaatacatattttt |
| | aaaacaaaccaataaatggcttacatga |
| SEQ ID NO: 3 | agacggcggcggcgcccgtaggatgcagggatcgctcccccggggccgctga |
| >NM_001099404.1 | gcctgcgcccagtgccccgagccccgcgccgagccgagtccgcgccaagcag |
| Homo sapiens | cagccgcccaccccggggcccggccgggggaccagcagcttccccacaggca |
| sodium voltage- | acgtgaggagagcctgtgcccagaagcaggatgagaagatggcaaacttcct |
| gated channel | attacctcggggcaccagcagcttccgcaggttcacacgggagtccctggca |
| alpha subunit 5 | gccatcgagaagcgcatggcagagaagcaagcccgcggctcaaccaccttgc |
| (SCN5A), | aggagagccgagaggggctgcccgaggaggaggctccccggccccagctgga |
| transcript variant | cctgcaggcctccaaaaagctgccagatctctatggcaatccaccccaagag |
| 3, mRNA | ctcatcggagagcccctggaggacctggaccccttctatagcacccaaaaga |
| [mRNA format is | ctttcatcgtactgaataaaggcaagaccatcttccggttcagtgccaccaa |
| obtained by | cgccttgtatgtcctcagtcccttccaccccatccggagagcggctgtgaag |
| exchanging | attctggttcactcgctcttcaacatgctcatcatgtgcaccatcctcacca |
| thymine (T) for | actgcgtgttcatggcccagcacgaccctccaccctggaccaagtatgtcga |
| uracil (U)] | gtacaccttcaccgccatttacacctttgagtctctggtcaagattctggct |
| | cgaggcttctgcctgcacgcgttcactttccttcgggacccatggaactggc |
| | tggactttagtgtgattatcatggcgtatgtatcagaaaatataaaactagg |
| | caatttgtcggctcttcgaactttcagagtcctgagagctctaaaaactatt |
| | tcagttatcccagggctgaagaccatcgtgggggccctgatccagtctgtga |
| | agaagctggctgatgtgatggtcctcacagtcttctgcctcagcgtctttgc |
| | cctcatcggcctgcagctcttcatgggcaacctaaggcacaagtgcgtgcgc |
| | aacttcacagcgctcaacggcaccaacggctccgtggaggccgacggcttgg |
| | tctgggaatccctggacctttacctcagtgatccagaaaattacctgctcaa |
| | gaacggcacctctgatgtgttactgtgtgggaacagctctgacgctgggaca |
| | tgtccggagggctaccggtgcctaaaggcaggcgagaaccccgaccacggct |
| | acaccagcttcgattcctttgcctgggcctttcttgcactcttccgcctgat |
| | gacgcaggactgctgggagcgcctctatcagcagaccctcaggtccgcaggg |
| | aagatctacatgatcttcttcatgcttgtcatcttcctggggtccttctacc |
| | tggtgaacctgatcctggccgtggtcgcaatggcctatgaggagcaaaacca |
| | agccaccatcgctgagaccgaggagaaggaaaagcgcttccaggaggccatg |
| | gaaatgctcaagaaagaacacgaggccctcaccatcaggggtgtggataccg |
| | tgtcccgtagctccttggagatgtcccctttggccccagtaaacagccatga |
| | gagaagaagcaagaggagaaaacggatgtcttcaggaactgaggagtgtggg |
| | gaggacaggctccccaagtctgactcagaagatggtcccagagcaatgaatc |
| | atctcagcctcacccgtggcctcagcaggacttctatgaagccacgttccag |
| | ccgcgggagcattttcacctttcgcaggcgagacctgggttctgaagcagat |
| | tttgcagatgatgaaaacagcacagcgggggagagcgagagccaccacacat |
| | cactgctggtgccctggcccctgcgccggaccagtgcccagggacagcccag |
| | tcccggaacctcggctcctggccacgccctccatggcaaaaagaacagcact |
| | gtggactgcaatggggtggtctcattactgggggcaggcgacccagaggcca |
| | catccccaggaagccacctcctccgccctgtgatgctagagcacccgccaga |
| | cacgaccacgccatcggaggagccaggcgggccccagatgctgacctcccag |
| | gctccgtgtgtagatggcttcgaggagccaggagcacggcagcgggccctca |
| | gcgcagtcagcgtcctcaccagcgcactggaagagttagaggagtctcgcca |
| | caagtgtccaccatgctggaaccgtctcgcccagcgctacctgatctgggag |
| | tgctgcccgctgtggatgtccatcaagcagggagtgaagttggtggtcatgg |
| | acccgtttactgacctcaccatcactatgtgcatcgtactcaacacactctt |
| | catggcgctggagcactacaacatgacaagtgaattcgaggagatgctgcag |
| | gtcggaaacctggtcttcacagggattttcacagcagagatgaccttcaaga |
| | tcattgccctcgacccctactactacttccaacagggctggaacatcttcga |
| | cagcatcatcgtcatccttagcctcatggagctgggcctgtcccgcatgagc |
| | aacttgtcggtgctgcgctccttccgcctgctgcgggtcttcaagctggcca |
| | aatcatggcccaccctgaacacactcatcaagatcatcgggaactcagtggg |
| | ggcactggggaacctgacactggtgctagccatcatcgtgttcatctttgct |
| | gtggtgggcatgcagctctttggcaagaactactcggagctgagggacagcg |
| | actcaggcctgctgcctcgctggcacatgatggacttctttcatgccttcct |
| | catcatcttccgcatcctctgtggagagtggatcgagaccatgtgggactgc |
| | atggaggtgtcggggcagtcattatgcctgctggtcttcttgcttgttatgg |
| | tcattggcaaccttgtggtcctgaatctcttcctggccttgctgctcagctc |
| | cttcagtgcagacaacctcacagcccctgatgaggacagagagatgaacaac |
| | ctccagctggccctggcccgcatccagaggggcctgcgctttgtcaagcgga |
| | ccacctgggatttctgctgtggtctcctgcggcagcggcctcagaagcccgc |
| | agcccttgccgcccagggccagctgcccagctgcattgccaccccctactcc |
| | ccgccacccccagagacggagaaggtgcctcccacccgcaaggaaacacggt |
| | ttgaggaaggcgagcaaccaggccagggcacccccggggatccagagcccgt |
| | gtgtgtgcccatcgctgtggccgagtcagacacagatgaccaagaagaagat |
| | gaggagaacagcctgggcacggaggaggagtccagcaagcagcaggaatccc |
| | agcctgtgtccggtggcccagaggcccctccggattccaggacctggagcca |
| | ggtgtcagcgactgcctcctctgaggccgaggccagtgcatctcaggccgac |
| | tggcggcagcagtggaaagcggaaccccaggccccagggtgcggtgagaccc |
| | cagaggacagttgctccgagggcagcacagcagacatgaccaacaccgctga |
| | gctcctggagcagatccctgacctcggccaggatgtcaaggacccagaggac |
| | tgcttcactgaaggctgtgtccggcgctgtccctgctgtgcggtggacacca |
| | cacaggccccagggaaggtctggtggcggttgcgcaagacctgctaccacat |
| | cgtggagcacagctggttcgagacattcatcatcttcatgatcctactcagc |
| | agtggagcgctggccttcgaggacatctacctagaggagcggaagaccatca |
| | aggttctgcttgagtatgccgacaagatgttcacatatgtcttcgtgctgga |
| | gatgctgctcaagtgggtggcctacggcttcaagaagtacttcaccaatgcc |
| | tggtgctggctcgacttcctcatcgtagacgtctctctggtcagcctggtgg |
| | ccaacaccctgggctttgccgagatgggccccatcaagtcactgcggacgct |
| | gcgtgcactccgtcctctgagagctctgtcacgatttgagggcatgagggtg |
| | gtggtcaatgccctggtgggcgccatcccgtccatcatgaacgtcctcctcg |
| | tctgcctcatcttctggctcatcttcagcatcatgggcgtgaacctctttgc |
| | ggggaagtttgggaggtgcatcaaccagacagagggagacttgcctttgaac |
| | tacaccatcgtgaacaacaagagccagtgtgagtccttgaacttgaccggag |
| | aattgtactggaccaaggtgaaagtcaactttgacaacgtgggggccgggta |
| | cctggcccttctgcaggtggcaacatttaaaggctggatggacattatgtat |
| | gcagctgtggactccagggggtatgaagagcagcctcagtgggaatacaacc |
| | tctacatgtacatctattttgtcattttcatcatctttgggtctttcttcac |
| | cctgaacctctttattggtgtcatcattgacaacttcaaccaacagaagaaa |
| | aagttagggggccaggacatcttcatgacagaggagcagaagaagtactaca |
| | atgccatgaagaagctgggctccaagaagccccagaagcccatcccacggcc |
| | cctgaacaagtaccagggcttcatattcgacattgtgaccaagcaggccttt |
| | gacgtcaccatcatgtttctgatctgcttgaatatggtgaccatgatggtgg |
| | agacagatgaccaaagtcctgagaaaatcaacatcttggccaagatcaacct |
| | gctctttgtggccatcttcacaggcgagtgtattgtcaagctggctgccctg |
| | cgccactactacttcaccaacagctggaatatcttcgacttcgtggttgtca |
| | tcctctccatcgtgggcactgtgctctcggacatcatccagaagtacttctt |
| | ctccccgacgctcttccgagtcatccgcctggcccgaataggccgcatcctc |
| | agactgatccgaggggccaaggggatccgcacgctgctctttgccctcatga |
| | tgtccctgcctgccctcttcaacatcgggctgctgctcttcctcgtcatgtt |
| | catctactccatctttggcatggccaacttcgcttatgtcaagtgggaggct |
| | ggcatcgacgacatgttcaacttccagaccttcgccaacagcatgctgtgcc |
| | tcttccagatcaccacgtcggccggctgggatggcctcctcagccccatcct |
| | caacactgggccgccctactgcgaccccactctgcccaacagcaatggctct |
| | cggggggactgcgggagcccagccgtgggcatcctcttcttcaccacctaca |
| | tcatcatctccttcctcatcgtggtcaacatgtacattgccatcatcctgga |
| | gaacttcagcgtggccacggaggagagcaccgagcccctgagtgaggacgac |
| | ttcgatatgttctatgagatctgggagaaatttgacccagaggccactcagt |
| | ttattgagtattcggtcctgtctgactttgccgatgccctgtctgagccact |
| | ccgtatcgccaagcccaaccagataagcctcatcaacatggacctgcccatg |
| | gtgagtggggaccgcatccattgcatggacattctctttgccttcaccaaaa |
| | gggtcctgggggagtctggggagatggacgccctgaagatccagatggagga |
| | gaagttcatggcagccaacccatccaagatctcctacgagcccatcaccacc |
| | acactccggcgcaagcacgaagaggtgtcggccatggttatccagagagcct |
| | tccgcaggcacctgctgcaacgctctttgaagcatgcctccttcctcttccg |
| | tcagcaggcgggcagcggcctctccgaagaggatgcccctgagcgagagggc |
| | ctcatcgcctacgtgatgagtgagaacttctcccgaccccttggcccaccct |
| | ccagctcctccatctcctccacttccttcccaccctcctatgacagtgtcac |
| | tagagccaccagcgataacctccaggtgcgggggtctgactacagccacagt |
| | gaagatctcgccgacttccccccttctccggacagggaccgtgagtccatcg |
| | tgtgagcctcggcctggctggccaggacacactgaaaagcagcctttttcac |
| | catggcaaacctaaatgcagtcagtcacaaaccagcctggggccttcctggc |
| | tttgggagtaagaaatgggcctcagccccgcggatcaaccaggcagagttct |
| | gtggcgccgcgtggacagccggagcagttggcctgtgcttggaggcctcaga |
| | tagacctgtgacctggtctggtcaggcaatgccctgcggctctggaaagcaa |
| | cttcatcccagctgctgaggcgaaatataaaactgagactgtatatgttgtg |
| | aatgggctttcataaatttattatatttgatatttttttacttgagcaaaga |
| | actaaggatttttccatggacatgggcagcaattcacgctgtctcttcttaa |
| | ccctgaacaagagtgtctatggagcagccggaagtctgttctcaaagcagaa |
| | gtggaatccagtgtggctcccacaggtcttcactgcccaggggtcgaatggg |
| | gtccccctcccacttgacctgagatgctgggagggctgaacccccactcaca |
| | caagcacacacacacagtcctcacacacggaggccagacacaggccgtggga |
| | cccaggctcccagcctaagggagacaggcctttccctgccggccccccaagg |
| | atggggttcttgtccacggggctcactctggccccctattgtctccaaggtc |
| | ccattttccccctgtgttttcacgcaggtcatattgtcagtcctacaaaaat |
| | aaaaggcttccagaggagagtggcctgggtcccagggctggccctaggcact |
| | gatagttgccttttcttcccctcctgtaagagtattaacaaaaccaaaggac |
| | acaagggtgcaagccccattcacggcctggcatgcagcttgtccttgctcct |
| | ggaacctggcaggccctgcccagccagccatcggaagagagggctgagccat |
| | gggggtttggggctaagaagttcaccagccctgagccatggcggcccctcag |
| | cctgcctgaagagaggaaactggcgatctcccagggctctctggaccatacg |
| | cggaggagttttctgtgtggtctccagctcctctccagacacagagacatgg |
| | gagtggggagcggagcttggccctgcgccctgtgcagggaaagggatggtca |
| | ggcccagttctcgtgcccttagaggggaatgaaccatggcacctttgagaga |
| | gggggcactgtggtcaggcccagcctctctggctcagcccgggatcctgatg |
| | gcacccacacagaggacctctttggggcaagatccaggtggtcccataggtc |
| | ttgtgaaaaggctttttcagggaaaaatattttactagtccaatcaccccca |
| | ggacctcttcagctgctgacaatcctatttagcatatgcaaatcttttaaca |
| | tagagaactgtcaccctgaggtaacagggtcaactggcgaagcctgagcagg |
| | caggggcttggctgccccattccagctctcccatggagcccctccaccgggc |
| | gcatgcctcccaggccacctcagtctcacctgccggctctgggctggctgct |
| | cctaacctacctcgccgagctgtcggagggctggacatttgtggcagtgctg |
| | aagggggcattgccggcgagtaaagtattatgtttcttcttgtcaccccagt |
| | tcccttggtggcaaccccagacccaacccatgcccctgacagatctagttct |
| | cttctcctgtgttccctttgagtccagtgtgggacacggtttaactgtccca |
| | gcgacatttctccaagtggaaatcctatttttgtagatctccatgctttgct |
| | ctcaaggcttggagaggtatgtgcccctcctgggtgctcaccgcctgctaca |
| | caggcaggaatgcggttgggaggcaggtcgggctgccagcccagctggccgg |
| | aaggagactgtggtttttgtgtgtgtggacagcccgggagctttgagacagg |
| | tgcctggggctggctgcagacggtgtggttgggggtgggaggtgagctagac |
| | ccaacccttagcttttagcctggctgtcacctttttaatttccagaactgca |
| | caatgaccagcaggagggaaggacagacatcaagtgccagatgttgtctgaa |
| | ctaatcgagcacttctcaccaaacttcatgtataaataaaatacatattttt |
| | aaaacaaaccaataaatggcttacatga |
| SEQ ID NO: 4 | 5′-CTGCACGCGTTCACTTTCCT-3′ |
| Forward primer, | |
| Neonatal Nav1.5 | |
| (SCN5A) | |
| SEQ ID NO: 5 | 5′-GACAAATTGCCTAGTTTTATATTT-3′ |
| Reverse primer, | |
| Neonatal Nav1.5 | |
| (SCN5A) | |
| SEQ ID NO: 6 | 5′-CTGCACGCGTTCACTTTCCT-3′ |
| Forward primer, | |
| general Nav1.5 | |
| (SCN5A) | |
| SEQ ID NO: 7 | 5′-CAGCCAGCTTCTTCACAGACT-3′ |
| Reverse primer, | |
| general Nav1.5 | |
| (SCN5A) | |
| SEQ ID NO: 8 | 5′-TATACACCCATCTCCAGCGA-3′ |
| Forward primer, | |
| control gene-1 | |
| (CYB5R3) | |
| SEQ ID NO: 9 | 5′-CATCTCCTCATTCACGAAGC-3′ |
| Reverse primer, | |
| control gene-1 | |
| (CYB5R3) | |
| SEQ ID NO: 10 | 5′-TGGGAACAAGAGGGCATCTG-3′ |
| Forward primer, | |
| control gene-2 | |
| (SDHA) | |
| SEQ ID NO: 11 | 5′-CCACCACTGCATCAAATTCATG-3′ |
| Reverse primer, | |
| control gene-2 | |
| (SDHA) | |
| SEQ ID NO: 12 | AGGUAGUGUAAUCGCCUUG |
| Control c-siRNA | |
| SEQ ID NO: 13 | CUAGGCAAUUUGUCGGCUC |
| Neo1 (n1-siRNA) | |
| SEQ ID NO: 14 | UAUCAUGGCGUAUGUAUCA |
| Neo2 (n2-siRNA) | |
| SEQ ID NO: 15 | GAGUCCUGAGAGCUCUAAA |
| NESO (n3-siRNA) | |
| SEQ ID NO: 16 | GUCUCAGCCUUACGCACCU |
| ADULT (a-siRNA) | |
| SEQ ID NO: 17 | GTGTTTAACCTGATTTTCACCTGAAATGACTGATATAGTTTTCAGGGCCCGG |
| GRCh38 | AGGACTCGGAAGGTGCGTAAGGCTGAGACATTGCCCAGGTCCACAAATTCAG |
| chromosome3 | TTGTGTATCTGTAACAAGGGAAATTCACACAGAGACAATGACAACACACCAA |
| position: | TAGGAGACACACAGTCAGAGGAG |
| 38613723-38614086, | |
| segment 1 | |
| SEQ ID NO: 18 | GAAAGGGGGTGGGGAAGACAGAGAGAGAGTCACTTGTAGCTGAGATCTGAGA |
| GRCh38 | GGCAAACCTGGGCATCTTACCTGGGATAACTGAAATAGTTTTTAGAGCTCTC |
| chromosome3 | AGGACTCTGAAAGTTCGAAGAGCCGACAAATTGCCTAGTTTTATATTTTCTG |
| position: | ATACATA CCTGCAGAATCAAACCACAGT (SEQ ID NO: 18) |
| 38613723-38614086, | |
| segment 2 | |
| SEQ ID NO: 19 | tacacaactgaatttgtggacctgggcaatgtctcagccttacgcaccttcc |
| Adult Nav1.5 | gagtcctccgggccctgaaaactatatcagtcatttca |
| DNA, exon 6, | |
| Ensembl exon | |
| accession: | |
| ENSE00001318072 | |
| SEQ ID NO: 20 | YTTEFVDLGNVSALRTFRVLRALKTISVIS |
| Adult aNav1.5, | |
| Ensembl exon | |
| accession: | |
| ENSE00001318072 | |
| SEQ ID NO: 21 | tatgtatcagaaaatataaaactaggcaatttgtcggctcttcgaactttca |
| Neonatal Nav1.5 | gagtcctgagagctctaaaaactatttcagttatccca |
| DNA, | |
| Ensembl exon | |
| accession: | |
| ENSE00001805777 | |
| SEQ ID NO: 22 | YVSENIKLGNLSALRTFRVLRALKTISVIP |
| SEQ ID NO: 23 | MANFLLPRGTSSFRRFTRESLAAIEKRMAEKQARGSTTLQESREGLPEEEAP |
| nNav1.5 (example) | RPQLDLQASKKLPDLYGNPPQELIGEPLEDLDPFYSTQKTFIVLNKGKTIFR |
| UniProtKB-H9KVD2 | FSATNALYVLSPFHPIRRAAVKILVHSLFNMLIMCTILTNCVFMAQHDPPPW |
| (H9KVD2_HUMAN) | TKYVEYTFTAIYTFESLVKILARGFCLHAFTFLRDPWNWLDFSVIIAMYVSE |
| >tr\|H9KVD2\|H9KVD2_ | NIKLGNLSALRTFRVLRALKTISVIPGLKTIVGALIQSVKKLADVMVLTVFC |
| HUMAN | LSVFALIGLQLFMGNLRHKCVRNFTALNGTNGSVEADGLVWESLDLYSDPEN |
| Sodium channel | YLLKNGTSDVLLCGNSSDAGTCPEGYRCLKAGENPDHGYTSFDSFAWAFLAL |
| protein | FRLMTQDCWERLYQQTLRSAGKIYMIFFMLVIFLGSFYLVNLILAVVAMAYE |
| OS = Homo sapiens | EQNQATIAETEEKEKRFQEAMEMLKKEHEALTIRGVDTVSRSSLEMSPLAPV |
| OX = 9606 | NSHERRSKRRKRMSSGTEECGEDRLPKSDSEDGPRMANHLSLTRGLSRTSMK |
| GN = SCN5A PE = 1 | PRSSRGSIFTFRRRDLGSEADFADDENSTAGESESHHTSLLVPWPLRRTSAQ |
| SV = 1 | GQPSPGTSAPGHALHGKKNSTVDCNGVVSLLGAGDPEATSPGSHLLRPVMLE |
| | HPPDTTTPSEEPGGPQMLTSQAPCVDGFEEPGARQRALSAVSVLTSALEELE |
| | ESRHKCPPCWNRLAQRYLIWECCPLWMSIKQGVKLVVMDPFTDLTITMCIVL |
| | NTLFMALEHYNMTSEFEEMLQVGNLVFTGIFTAEMTFKIIALDPYYYFQQGW |
| | NIFDSIIVILSLMELGLSRMSNLSVLRSFRLLRVFKLAKSWPTLNTLIKIIG |
| | NSVGALGNLTLVLAIIVFIFAVVGMQLFGKNYSELRDSDSGLLPRWHMMDFF |
| | HAFLIIFRILCGEWIETMWDCMEVSGQSLCLLVFLLVMVIGNLVVLNLFLAL |
| | LLSSFSADNLTAPDEDREMNNLQLALARIQRGLRFVKRTTWDFCCGLLRQRP |
| | QKPAALAAQGQLPSCIATPYSPPPPETEKVPPTRKETRFEEGEQPGQGTPGD |
| | PEPVCVPIAVAESDTDDQEEDEENSLGTEEESSKQQESQPVSGGPEAPPDSR |
| | TWSQVSATASSEAEASASQADWRQQWKAEPQAPGCGETPEDSCSEGSTADMT |
| | NTAELLEQIPDLGQDVKDPEDCFTEGCVRRCPCCAVDTTQAPGKVWWRLRKT |
| | CYHIVEHSWFETFIIFMILLSSGALAFEDIYLEERKTIKVLLEYADKMFTYV |
| | FVLEMLLKWVAYGFKKYFTNAWCWLDFLIVDVSLVSLVANTLGFAEMGPIKS |
| | LRTLRALRPLRALSRFEGMRVVVNALVGAIPSIMNVLLVCLIFWLIFSIMGV |
| | NLFAGKFGRCINQTEGDLPLNYTIVNNKSQCESLNLTGELYWTKVKVNFDNV |
| | GAGYLALLQVATFKGWMDIMYAAVDSRGYEEQPQWEYNLYMYIYFVIFIIFG |
| | SFFTLNLFIGVIIDNFNQQKKKLGGQDIFMTEEQKKYYNAMKKLGSKKPQKP |
| | IPRPLNKYQGIFIFDIVTKQAFDVTIMFLICLNMVTMMVETDDQSPEKINIL |
| | AKINLLFVAIFTGECIVKLAALRHYYFTNSWNIFDFVVVILSIVGTVLSDII |
| | QKYFFSPTLFRVIRLARIGRILRLIRGAKGIRTLLFALMMSLPALFNIGLLL |
| | FLVMFIYSIFGMANFAYVKWEAGIDDMFNFQTFANSMLCLFQITTSAGWDGL |
| | LPILNTGPPYCDPTLPNSNGSRGDCGSPAVGILFFTTYIIISFLIVVNMYIA |
| | IILENFSVATEESTEPLSEDDFDMFYEIWEKFDPEATQFIEYSVLSDFADAL |
| | SEPLRIAKPNQISLINMDLPNVSGDRIHCMDILFAFTKRVLGESGEMDALKI |
| | QMEEKFMAANPSKISYEPITTTLRRKHEEVSAMVIQRAFRRHLLQRSLKHAS |
| | FLFRQQAGSGLSEEDAPEREGLIAYVMSENFSRPLGPPSSSSISSTSFPPSY |
| | DSVTRATSDNLQVRGSDYSHSEDLADFPPSPDRDRESIV |
| SEQ ID NO: 24 | YVTEFVXLGNVSALRTFRVLRALKTISVIP, wherein “X” is K or |
| Partial consensus | D |
| sequence | |
| nNav1.5 and aNav1.5 | |
| (FIG. 12) | |

### LIST OF REFERENCES

Each reference cited below or elsewhere in the present application is hereby incorporated by reference, in its entirety.

Andrikopoulos et al., The Journal Of Biological Chemistry Vol. 286, No. 19, pp. 16846-16860, May 13, 2011

Antzelevitch C, Belardinelli L, Zygmunt A C, Burashnikov A, Di Diego J M, Fish J M, Cordeiro J M, Thomas G. 2004. Electrophysiological effects of ranolazine, a novel antianginal agent with antiarrhythmic properties. Circulation 110:904-910.

Banskota S, Regmi S C, Kim J-A. 2015. NOX1 to NOX2 switch deactivates AMPK and induces invasive phenotype in colon cancer cells through overexpression of MMP-7. Mol Cancer 14:123.

Baptista-Hon D T, Robertson F M, Robertson G B, Owen S J, Rogers G W, Lydon E L, Lee N H, Hales T G. 2014. Potent inhibition by ropivacaine of metastatic colon cancer SW620 cell invasion and Nav1.5 channel function. Br J Anaesthes 113:i39-i48.

Belardinelli L, Shryock J C, Fraser, H. 2006. Inhibition of the late sodium current as a potential cardioprotective principle: effects of the late sodium current inhibitor ranolazine. Heart 92:6-14.

Bennett E S, Smith B A, Harper J M. 2004. Voltage-gated Na+ channels confer invasive properties on human prostate cancer cells. Pflugers Arch 447:908-914.

Ben-Porath I, Thomson M W, Carey V J, Ge R, Bell G W, Regev A, Weinberg R A. 2008. An embryonic stem cell-like gene expression signature in poorly differentiated aggressive human tumors. Nature Genetics 40:499-507.

Brackenbury W J, Chioni A M, Diss J K J, Djamgoz M B A. 2007. The neonatal splice variant of Nav1.5 potentiates in vitro invasive behaviour of MDA-MB-231 human breast cancer cells. Breast Cancer Res Treat 101:149-160.

Brackenbury W J, Djamgoz M B A. 2006. Activity-dependent regulation of voltage-gated Na+ channel expression in Mat-LyLu rat prostate cancer cell line. J Physiol 573:343-356.

Brattain M G, Fine W D, Khaled F M, Thompson J, Brattain D E. 1981. Heterogeneity of malignant cells from a human colonic carcinoma. Cancer Res 41:1751-1756.

Campbell T M, Main M J, Fitzgerald E M. 2013. Functional expression of the voltage-gated Na⁺-channel Nav1.7 is necessary for EGF-mediated invasion in human non-small cell lung cancer cells. J Cell Sci 126:4939-4949. Chioni A M, Fraser S P, Pani F, Foran P, Wilkin G P, Diss J K J, Djamgoz M B A. 2005. A novel polyclonal antibody specific for the Nav1.5 voltage-gated Na+ channel ‘neonatal’ splice form. J Neurosci Methods 147:88-98.

Djamgoz M B A, Onkal R. 2013. Persistent current blockers of voltage-gated sodium channels: a clinical opportunity for controlling metastatic disease. Recent Pat Anticancer Drug Discov 8:66-84.

Djamgoz M B A. 2011. Bioelectricity of cancer: Voltage-gated ion channels and direct-current electric fields. In The Physiology of Bioelectricity in Development, Tissue Regeneration, and Cancer (Ed C Pullar). Taylor & Francis (London and NY). Pp. 269-294.

Driffort V, Gillet L, Bon E, Marionneau-Lambot S, Oullier T, Joulin V, Collin C, Pages J C, Jourdan M L, Chevalier S, Bougnoux P, Le Guennec J Y, Besson P, Roger S. 2014. Ranolazine inhibits Nav1.5-mediated breast cancer cell invasiveness and lung colonization. Mol Cancer 13: 264.

Elajnaf et al., Anesth Analg. 2018 September; 127(3):650-660

Fearon E R. 2011. Molecular genetics of colorectal cancer. Annu Rev Pathol 6:479-507.

Fitzsimmons S A, Workman P, Greyer M, Paull K, Camalier R, Lewis A D. 1996. Reductase enzyme expression across the National Cancer Institute Tumor cell line panel: correlation with sensitivity to mitomycin C and EO9. J Natl Cancer Inst 88:259-269.

Fogh J. 1975. Human tumor cells in vitro. Plenum Press: New York.

Fraser S P, Ding Y, Liu A, Foster C S, Djamgoz M B A. 1999. Tetrodotoxin suppresses morphological enhancement of the metastatic MAT-LyLu rat prostate cancer cell line. Cell Tissue Res 295:505-512.

Fraser S P, Diss J K J, Chioni A M, Mycielska M E, Pan H, Yamaci R F, Pani F, Siwy Z, Krasowska M, Grzywna Z, Brackenbury W J, Theodorou D, Koyuturk M, Kaya H, Battaloglu E, De Bella M T, Slade M J, Tolhurst R, Palmieri C, Jiang J, Latchman D S, Coombes R C, Djamgoz M B A. 2005. Voltage-gated sodium channel expression and potentiation of human breast cancer metastasis. Clin Cancer Res 11:5381-5389.

Fraser S P, Grimes J A, Diss J K J, Stewart D, Dolly J O, Djamgoz M B A. 2003a. Predominant expression of Kv1.3 voltage-gated K+ channel subunit in rat prostate cancer cell lines: electrophysiological, pharmacological and molecular characterisation. Pflugers Arch 446:559-571.

Fraser S P, Salvador V, Manning E, Mizal J, Altun S, Reza M, Berridge R J, Djamgoz M B A. 2003b. Contribution of functional voltage-gated Na+ channel expression to cell behaviours involved in the metastatic cascade in rat prostate cancer: I. Lateral motility. J Cell Physiol 195:479-487.Gao, R.; Shen, Y.; Cai, J.; Lei, M.; Wang, Z. 2010. Expression of voltage-gated sodium channel alpha subunit in human ovarian cancer. Oncol. Rep. 23, 1293-1299.

Gao, R.; Cao, T.; Chen, H.; Cai, J.; Lei, M.; Wang, Z. 2019. Nav1.5-E3 antibody inhibits cancer progression. Transl. Cancer Res. 8, 44-50.

Gillet L, Roger S, Besson P, Lecaille F, Gore J, Bougnoux P, Lalmanach G, Le Guennec J-Y. 2009. Voltage-gated sodium channel activity promotes cysteine cathepsin-dependent invasiveness and colony growth of human cancer cells. J Biol Chem 284:8680-8691.

Grimes J A, Fraser S P, Stephens G J, Downing J E, Laniado M E, Foster C S, Abel P D, Djamgoz M B A. 1995. Differential expression of voltage-activated Na+ currents in two prostatic tumour cell lines: contribution to invasiveness in vitro. FEBS Lett 369:290-294.

Guzel, R 2012. Studies of ionic mechanisms associated with human cancers. Ph.D. Thesis, Imperial College London.

Guzel, R. M.; Ogmen, K.; Ilieva, K. M.; Fraser, S. P.; Djamgoz, M. B. A. 2019. Colorectal cancer invasiveness in vitro: Predominant contribution of neonatal Nav1.5 under normoxia and hypoxia. J. Cell. Physiol. 234, 6582-6593.Hongo K, Tsuno NH, Kawai K, Sasaki K, Kaneko M, Hiyoshi M, Murono K, Tada N, Nirei T, Sunami E, Takahashi K, Nagawa H, Kitayama Watanabe T. 2013. Hypoxia enhances colon cancer migration and invasion through promotion of epithelial-mesenchymal transition. J Surg Res 182:75-84.

House C D, Vase C J, Schwartz A M, Obias V, Frank B, Luu T, Sarvazyan N, Irby R, Strausberg R L, Hales T G, Stuart J M, Lee N H. 2010. Voltage-gated Na+ channel SCNSA is a key regulator of a gene transcriptional network that controls colon cancer invasion. Cancer Res 70:6957-6967.

House C D, Wang B D, Ceniccola K, Williams R, Simaan M, Olender J, Patel V, Baptista-Hon D T, Annunziata C M, Gutkind J S, Hales T G, Lee N H. 2015. Voltage-gated Na+ channel activity increases colon cancer transcriptional activity and invasion via persistent MAPK signaling. Sci Rep 5:11541.

Jacob F, Guertler R, Naim S, Nixdorf S, Fedier A, Hacker N F, Heinzelmann-Schwarz V. 2013. Careful selection of reference genes is required for reliable performance of RT-qPCR in human normal and cancer cell lines. PLoS One. 8:e59180.

Ju Y K, Saint D A, Gage P W. 1996. Hypoxia increases persistent sodium current in rat ventricular myocytes. J Physiol 497:337-347.

Karsa L V, Lignini T A, Patnick J, Lambert R, Sauvaget C. 2010. The dimensions of the CRC problem. Best Pract Res Clin Gastroenterol 24:381-396.

Kawamoto H, Yuasa T, Kubota Y, Seita M, Sasamoto H, Shahid J M, Hayashi T, Nakahara H, Hassan R, Iwamuro M, Kondo E, Nakaji S, Tanaka N, Kobayashi N. 2010. Characteristics of CD133(+) human colon cancer SW620 cells. Cell Transplant 19:857-864.

Koltai T. 2015. Voltage-gated sodium channel as a target for metastatic risk reduction with re-purposed drugs. F1000Res 4:297.

Krishnamachary B, Berg-Dixon S, Kelly B, Agani F, Feldser D, Ferreira G, Iyer N, LaRusch J, Pak B, Taghavi P, Semenza G L. 2003. Regulation of colon carcinoma cell invasion by hypoxia-inducible factor 1. Cancer Res 63:1138-43.

Laniado M E, Fraser S P, Djamgoz M B A. 2001. Voltage-gated K+ channel activity in human prostate cancer cell lines of markedly different metastatic potential: Distinguishing characteristics of PC-3 and LNCaP cells. The Prostate 46:262-274.

Laniado M E, Lalani E-N, Fraser S P, Grimes J A, Bhangal G, Djamgoz M B A, Abel P D. 1997. Expression and functional analysis of voltage-activated Na+ channels in human prostate cancer cell lines and their contribution to invasion in vitro. Am J Pathol 150:1213-1221.

Leibovitz A, Stinson J C, McCombs W B, McCoy C E, Mazur K C, Mabry N D. 1976. Classification of human colorectal adenocarcinoma cell lines. Cancer Res 36:4562-4569.

Livak K J, Schmittgen T D. 2001. Analysis of relative gene expression data using real-time quantitative PCR and the 2(-Delta Delta C(T)) Method. Methods 25:402-408.

Marin A, Lopez de Cerain A, Hamilton E, Lewis A D, Martinez-Penuela J M, Idoate M A, Bello J. 1997. DT-diaphorase and cytochrome B5 reductase in human lung and breast tumours. Br J Cancer 76:923-929.

Martin, K. C.; Zukin, R. S. RNA trafficking and local protein synthesis in dendrites: an overview. J. Neurosci. 2006, 26, 7131-7134.

McKeown S R. 2014. Defining normoxia, physoxia and hypoxia in tumours-implications for treatment response. Br J Radiol 87:20130676.

Nelson M, Yang M, Millican-Slater R, Brackenbury W J. 2015. Nav1.5 regulates breast tumor growth and metastatic dissemination in vivo. Oncotarget 6:32914-32929.

Ogino S, Chan A T, Fuchs C S, Giovannucci E. 2011. Molecular pathological epidemiology of colorectal neoplasia: An emerging transdisciplinary and interdisciplinary field. Gut 60:397-411.

Onkal R, Mattis J H, Fraser S P, Diss J K J, Shao D, Okuse K, Djamgoz M B A. 2008. Alternative splicing of Nav1.5: An electrophysiological comparison of ‘neonatal’ and ‘adult’ isoforms, and critical involvement of a lysine residue. J Cell Physiol 216:716-726.

Ou, S. W.; Kameyama, A.; Hao, L. Y.; Horiuchi, M.; Minobe, E.; Wang, W. Y.; Makita, N.; Kameyama, M. 2005. Tetrodotoxin-resistant Na+ channels in human neuroblastoma cells are encoded by new variants of Nav1.5/SCN5A. Eur. J. Neurosci. 22, 793-801.

Ouwerkerk R, Jacobs M A, Macura K J, Wolff A C, Stearns V, Mezban S D, Khouri N F, Bluemke D A, Bottomley P A. 2007. Elevated tissue sodium concentration in malignant breast lesions detected with non-invasive 23Na MRI. Breast Cancer Res Treat 106:151-160.

Peng J, Ou Q, Wu X, Zhang R, Zhao Q, Jiang W, Lu Z, Wan D, Pan Z, Fang Y. 2017. Expression of voltage-gated sodium channel Nav1.5 in non-metastatic colon cancer and its associations with estrogen receptor (ER)-β expression and clinical outcomes. Chin J Cancer 36:89.

Roger S, Besson P, Le Guennec J Y. 2003. Involvement of a novel fast inward sodium current in the invasion capacity of a breast cancer cell line. Biochim Biophys Acta 1616:107-111.

Roger S, Rollin J, Barascu A, Besson P, Raynal P I, Iochmann S, Lei M, Bougnoux P, Gruel Y, Le Guennec J Y. 2007. Voltage-gated sodium channels potentiate the invasive capacities of human non-small-cell lung cancer cell lines. Int J Biochem Cell Biol 39:774-786.

Tatrai E, Bartal A, Gacs A, Paku S, Kenessey I, Garay T, Hegedus B, Molnar E, Cserepes M T, Hegedus Z, Kucsma N, Szakacs G, Tovari J. 2017. Cell type-dependent HIF1 alpha-mediated effects of hypoxia on proliferation, migration and metastatic potential of human tumor cells. Oncotarget 8:44498-44510.

Tian Q, Stepaniants S B, Mao M, Weng L, Feetham M C, Doyle M J, Yi E C, Dai H, Thorsson V, Eng J, Goodlett D, Berger J P, Gunter B, Linseley P S, Stoughton R B, Aebersold R, Collins S J, Hanlon W A, Hood L E. 2004. Integrated genomic and proteomic analyses of gene expression in mammalian cells. Mol Cell Proteomics 3:960-969.

Torre L A, Siegel R L, Ward E M, Jemal A. 2016. Global cancer incidence and mortality rates and trends—An update. Cancer Epidemiol Biomarkers Prey 25:16-27.

Van Emburgh B O, Sartore-Bianchi A, Di Nicolantonio F, Siena S, Bardelli A. 2014. Acquired resistance to EGFR-targeted therapies in colorectal cancer. Mol Oncol 8:1084-1094.

Xie, A.; Gallant, B.; Guo, H.; Gonzalez, A.; Clark, M.; Madigan, A.; Feng, F.; Chen, H. D.; Cui, Y.; Dudley, S. C. Jr.; Wan, Y. 2018. Functional cardiac Na+ channels are expressed in human melanoma cells. Oncol. Lett. 16, 1689-1695.

Xing, D.; Wang, J.; Ou, S.; Wang, Y.; Qiu, B.; Ding, D.; Guo, F.; Gao, Q. 2014. Expression of neonatal Nav1.5 in human brain astrocytoma and its effect on proliferation, invasion and apoptosis of astrocytoma cells. Oncol. Rep. 31, 2692-700.

Yamaci R F, Fraser S P, Battaloglu E, Kaya H, Erguler K, Foster C S, Djamgoz, M B A. 2017. Neonatal Nav1.5 protein expression in normal adult human tissues and breast cancer. Pathol Res ract 213:900-907.

Yildirim S, Altun S, Gumushan H, Patel A, Djamgoz M B A. 2012. Voltage-gated sodium channel activity promotes prostate cancer metastasis in vivo. Cancer Lett 323:58-61.

You Y N, Xing Y, Feig B W, Chang G J, Cormier J N. 2012. Young-onset colorectal cancer: Is it time to pay attention? Arch Intern Med 172:287-289.

Young P E, Womeldorph C M, Johnson E K, Maykel J A, Brucher B, Stojadinovic A, Avital I, Nissan A, Steele S R. 2014. Early detection of colorectal cancer recurrence in patients undergoing surgery with curative intent: Current status and challenges. J Cancer 5:262-271.

Zhang B, Wang J, Wang X, Zhu J, Liu Q, Shi Z, Chambers M C, Zimmerman L J, Shaddox K F, Kim S, Davies S R, Wang S, Wang P, Kinsinger C R, Rivers R C, Rodriguez H, Townsend R R, Ellis M J C, Carr S A, Tabb D L, Coffey R J, Slebos R J C, Liebler D C and NCI CPTAC Investigators. 2014. Proteogenomic characterization of human colon and rectal cancer. Nature 513:382-387.

Zhang, J.; Mao, W.; Dai, Y.; Qian, C.; Dong, Y.; Chen, Z.; Meng, L.; Jiang, Z.; Huang, T.; Hu, J.; Luo, P.; Korner, H.; Jiang, Y.; Ying, S. 2019. Voltage-gated sodium channel Nav1.5 promotes proliferation, migration and invasion of oral squamous cell carcinoma. Acta Biochim. Biophys. Sin. (Shanghai) 51,562-570.

WO 2018/146313 (Celex GmbH)

WO2012/049440 (Celex Oncology Ltd.)

1. A method of treating a cancer comprising cancer cells that express the neonatal form of human Nav1.5 (nNav1.5), comprising administering to a subject suffering from said cancer an oligomeric compound comprising a target binding domain that is specifically hybridisable to mRNA or genomic DNA encoding nNav1.5, wherein the oligomeric compound reduces the level of mRNA encoding nNav1.5 in the cancer cells, the level of nNav1.5 in the cancer cells and/or the level of nNav1.5 expressed on the surface of the cancer cells.

2. The method of claim 1, wherein the cancer is colorectal cancer, breast cancer, lung cancer, ovarian cancer, astrocytoma or neuroblastoma, or a combination of any thereof.

3. The method of claim 2, wherein the cancer is colorectal cancer.

4. The method of claim 1, wherein the target binding domain is specifically hybridisable to mRNA encoding nNav1.5.

5. The method of claim 1, wherein the nNav1.5 comprises a Lys (K) in position 211 of SEQ ID NO:1.

6. The method of claim 5, wherein the nNav1.5 comprises the amino acids V, S, N, I, K, L, and Pin positions 206, 207, 209, 210, 211, 215, and 234 of SEQ ID NO:1, respectively.

7. The method of claim 1, wherein the mRNA comprises a segment at least about 90%, such as at least about 95%, such as at least about 96%, 97%, 98%, 99% or 100% identical to a sequence directly complementary to SEQ ID NO:21.

8. The method of claim 1, wherein the target binding domain, the oligomeric compound, or both, is a 15, 16, 17, 18, 19, 20, 21, 22, 23, 24, 25, 26, 27, 28, 29 or 30-mer, optionally in double-stranded form.

9. The method of claim 1, wherein the target binding domain, the oligomeric compound, or both, is a ribonucleic acid (RNA), deoxyribonucleic acid (DNA), peptide nucleic acid (PNA), locked nucleic acid (LNA), unlocked nucleic acid (UNA), a phosphorodiamidate Morpholino oligomer (PMO) molecule, or a combination of any two or more thereof.

10. The method of claim 9, wherein the oligomeric compound is comprised in or encoded by a vector, such as a viral vector, optionally wherein the vector further comprises one or more expression control sequences.

11. The method of claim 10, wherein the vector further comprises a transactivating crRNA (tracrRNA), a nucleic acid encoding a CRISPR-associated enzyme selected from Cas9 and Cpf1, or both.

12. The method of claim 9, wherein the target binding domain, the oligomeric compound, or both, is an RNA molecule selected from an small interfering RNA (siRNA), short hairpin RNA(shRNA), a guide RNA (gRNA), single guide RNA (sgRNA), or CRISPR RNA (crRNA) molecule.

13. The method of claim 12, wherein the oligomeric compound is an siRNA molecule, optionally in double-stranded form.

14. The method of claim 12, wherein the target-binding domain is specifically hybridisable or directly complementary to a contiguous portion of residues 797 to 896 of SEQ ID NO:3.

15. The method of claim 12, wherein the target binding domain is specifically hybridisable or directly complementary to genomic DNA transcribed into GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or to two or all of SEQ ID NOS:13-15, or to mRNA transcribed therefrom.

16. The method of claim 12, wherein the oligomeric compound comprises or consists of the RNA sequence (in 5′→3′ direction) GAGUCCUGAGAGCUCUAAA (NESO; SEQ ID NO:15);
CUAGGCAAUUUGUCGGCUC (Neo1; SEQ ID NO:13), UAUCAUGGCGUAUGUAUCA (Neo2; SEQ ID NO:14), or a combination of two or all thereof, optionally in double-stranded form.

17. The method of claim 1, wherein the oligomeric compound is comprised in a lipid nanoparticle (LNP) or liposome.

18. The method of claim 1, wherein the method reduces or prevents metastatic behaviour of the cancer, pain sensation in the subject, invasiveness of the cancer, overall aggressiveness of the cancer, or any combination thereof.

19. The method of claim 1, comprising determining that the cancer expresses nNav1.5 prior to administering the oligomeric compound.

20. The method of claim 1, wherein the cancer comprises one or more hypoxic tumours.

21. The method of claim 1, comprising administering a second therapeutic agent to the subject.

22. The method of claim 21, wherein the second therapeutic agent is not a VGSC blocker.

23. A method of treating a cancer selected from colorectal cancer, breast cancer, lung cancer, ovarian cancer or neuroblastoma, or a combination of any thereof, wherein the oligomeric compound comprises a target binding domain that is specifically hybridisable to messenger RNA (mRNA) or genomic DNA encoding nNav1.5.

24. An isolated oligomeric compound comprising or consisting of the RNA sequence GAGUCCUGAGAGCUCUAAA (NESO; SEQ ID NO:15); CUAGGCAAUUUGUCGGCUC (Neo1; SEQ ID NO:13), UAUCAUGGCGUAUGUAUCA (Neo2; SEQ ID NO:14), or an RNA sequence directly complementary to SEQ ID NO:15, SEQ ID NO:13 or SEQ ID NO:14 in double-stranded form with a complementary RNA sequence.
